(19) United States
(12) Patent Application Publication
(10) Pub. No.: US 2007/0049563 A1
(43) Pub. Date: Mar. 1, 2007

(54) Tigecycline and methods of preparing 9-aminominocycline

(76) Inventors: Lalitha Krishnan, Suffern, NY (US); Phaik-Eng Sum, Pomona, NY (US); Sylvain Daigneault, Laval (CA); Michel Bernatchez, Montreal-nord (CA); Anthony Scott Pilcher, Colchester, VT (US); Jeffrey Marshall Horne, Mooers, NY (US); Adam Joseph Tuper, Plattsburgh, NY (US); Joseph James McCauley III, Plattsburgh, NY (US); Adam P. Michaud, Lawrenceville, NJ (US)

Correspondence Address: WYETH/FINNEGAN HENDERSON, LLP, 901 NEW YORK AVENUE, NW, WASHINGTON, DC, 20001-4413, US

(21) Appl. No.: 11/440,038

(22) Filed: May 25, 2006

Related U.S. Application Data
(60) Provisional application No. 60/685,146, filed on May 27, 2005.

(51) Int. Cl. A61K 31/65; C07C 237/26

(52) U.S. Cl. 514/152; 552/205

(57) ABSTRACT

Methods of preparing and purifying tetracyclines, such as tigecycline, are disclosed. Also disclosed are tetracycline compositions, such as tigecycline compositions, prepared by these methods.

This application claims benefit of U.S. Provisional Application No. 60/685,146, filed May 27, 2005, the contents of which are incorporated herein by reference.

Disclosed herein are methods of preparing at least one compound of formula 1,

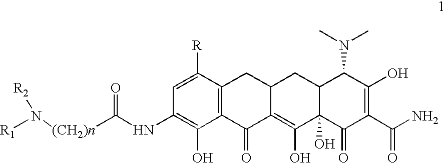

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4.

In one embodiment, R₁ is hydrogen, R₂ is t-butyl, R is —NR₃R₄ where R₃ is methyl and R₄ is methyl, and n is 1, for example, tigecycline. Tigecycline, (9-(t-butyl-glycylamido)-minocycline, TBA-MINO), (4S,4aS,5aR,12aS)-9-[2-(tert-butylamino)acetamido]-4,7-bis(dimethylamino)-1,4,4a,5,5a,6,11,12a-octahydro-3,10,12,12a-tetrahydroxy-1,11-dioxo-2-naphthacenecarboxamide, where R₁ is hydrogen, R₂ is t-butyl, R₃ is methyl, R₄ is methyl, and n is 1. Tigecycline is a glycylcycline antibiotic and an analog of the semisynthetic tetracycline, minocycline. Tigecycline is a 9-t-butylglycylamido derivative of minocycline, as shown in the structure below:

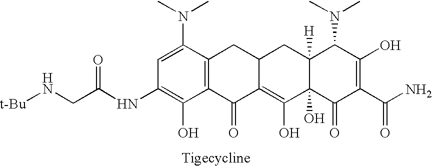

Tigecycline was developed in response to the worldwide threat of emerging resistance to antibiotics. Tigecycline has expanded broad-spectrum antibacterial activity both in vitro and in vivo. Glycylcycline antibiotics, like tetracycline antibiotics, act by inhibiting protein translation in bacteria.

Tigecycline is a known antibiotic in the tetracycline family and a chemical analog of minocycline. It may be used as a treatment against drug-resistant bacteria, and it has been shown to work where other antibiotics have failed. For example, it is active against methicillin-resistant Staphylococcus aureus, penicillin-resistant Streptococcus pneumoniae, vancomycin-resistant enterococci (D. J. Beidenbach et. al., Diagnostic Microbiology and Infectious Disease 40:173-177 (2001); H. W. Boucher et. al., Antimicrobial Agents & Chemotherapy 44:2225-2229 (2000); P. A. Bradford Clin. Microbiol. Newslett. 26:163-168 (2004); D. Milatovic et. al., Antimicrob. Agents Chemother. 47:400-404 (2003); R. Patel et. al., Diagnostic Microbiology and Infectious Disease 38:177-179 (2000); P. J. Petersen et. al., Antimicrob. Agents Chemother. 46:2595-2601 (2002); and P. J. Petersen et. al., Antimicrob. Agents Chemother. 43:738-744(1999), and against organisms carrying either of the two major forms of tetracycline resistance: efflux and ribosomal protection (C. Betriu et. al., Antimicrob. Agents Chemother. 48:323-325 (2004); T. Hirata et. al. Antimicrob. Agents Chemother. 48:2179-2184 (2004); and P. J. Petersen et. al., Antimicrob. Agents Chemother. 43:738-744(1999).

Tigecycline may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, tigecycline may be used to treat or control bacterial infections in warm-blooded animals caused by bacteria having the TetM and TetK resistant determinants. Also, tigecycline may be used to treat bone and joint infections, catheter-related Neutropenia, obstetrics and gynecological infections, or to treat other resistant pathogens, such as VRE, ESBL, enterics, rapid growing mycobacteria, and the like.

Tigecycline suffers some disadvantages in that it may degrade by epimerization. Epimerization is a known degradation pathway in tetracyclines generally, although the rate of degradation may vary depending upon the tetracycline. Comparatively, the epimerization rate of tigecycline may be fast, even for example, under mildly acidic conditions and/or at mildly elevated temperatures. The tetracycline literature reports several methods scientists have used to try and minimize epimer formation in tetracyclines. In some methods, the formation of calcium, magnesium, zinc or aluminum metal salts with tetracyclines limit epimer formation when done at basic pHs in non-aqueous solutions. (Gordon, P. N, Stephens Jr, C. R., Noseworthy, M. M., Teare, F. W., U.K. Patent No. 901,107). In other methods, (Tobkes, U.S. Pat. No. 4,038,315) the formation of a metal complex is performed at acidic pH and a stable solid form of the drug is subsequently prepared.

Tigecycline differs structurally from its epimer in only one respect.

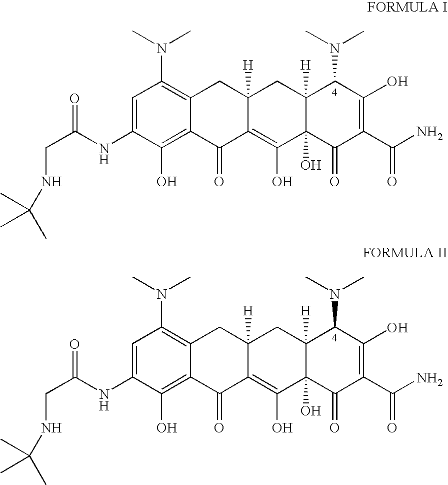

In tigecycline, the N-dimethyl group at the 4 carbon is cis to the adjacent hydrogen as shown above in formula I, whereas in the epimer (i.e., the C₄-epimer), formula II, they are trans to one another in the manner indicated. Although the tigecycline epimer is believed to be non-toxic, under certain conditions it may lack the anti-bacterial efficacy of tigecycline and may, therefore, be an undesirable degradation product. Moreover, the amount of epimerization can be magnified when synthesizing tigecycline in a large scale.

Other methods for reducing epimer formation include maintaining pHs of greater than about 6.0 during processing; avoiding contact with conjugates of weak acids such as formates, acetates, phosphates, or boronates; and avoiding contact with moisture including water-based solutions. With regard to moisture protection, Noseworthy and Spiegel (U.S. Pat. No. 3,026,248) and Nash and Haeger, (U.S. Pat. No. 3,219,529) have proposed formulating tetracycline analogs in non-aqueous vehicles to improve drug stability. However, most of the vehicles included in these disclosures are more appropriate for topical than parenteral use. Tetracycline epimerization is also known to be temperature dependent so production and storage of tetracyclines at low temperatures can also reduce the rate of epimer formation (Yuen, P. H., Sokoloski, T. D., J. Pharm. Sci. 66:1648-1650, 1977; Pawelczyk, E., Matlak, B, Pol. J. Pharmacol. Pharm. 34: 409-421, 1982). Several of these methods have been attempted with tigecycline but apparently none have succeeded in reducing both epimer formation and oxidative degradation while not introducing additional degradants. Metal complexation, for example, was found to have little affect on either epimer formation or degradation generally at basic pH.

Although the use of phosphate, acetate, and citrate buffers improve solution state stability, they seem to accelerate degradation of tigecycline in the lyophilized state. Even without a buffer, however, epimerization is a more serious problem with tigecycline than with other tetracyclines such as minocycline.

In addition to the C₄-epimer, other impurities include oxidation by-products. Some of these by-products are obtained by oxidation of the D ring of the molecule, which is an aminophenol. Compounds of formula 3 (see Scheme I below) can be readily oxidized at the C-11 and C-12a positions. Isolation of compounds of formula 3 by precipitation with a non-solvent can have the problem that oxidation by-products and metal salts coprecipitate with the product resulting in very low purities. The oxidation and degradation of the nucleus of compounds of formula 3 can be more pronounced under basic reaction conditions and more so on large-scale operations since processing times are typically longer and the compounds are in contact with the base for a longer time.

Moreover, degradation products may be obtained during each of the different synthetic steps of a scheme, and separating the required compound from these degradation products can be tedious. For example, conventional purification techniques, such as chromatography on silica gel or preparative HPLC cannot be used to purify these compounds easily because of their chelating properties. Although some tetracyclines have been purified by partition chromatography using columns made of diatomaceous earth impregnated with buffered stationary phases containing sequestering agents like EDTA, these techniques can suffer from very low resolution, reproducibility and capacity. These disadvantages may hamper a large-scale synthesis. HPLC has also been used for purification, but adequate resolution of the various components on the HPLC columns requires the presence of ion-pairing agents in the mobile phase. Separating the final product from the sequestering and ion-pairing agents in the mobile phase can be difficult.

While on a small-scale the impure compounds obtained by precipitation may be purified by preparative reverse-phase HPLC, purification by reverse phase liquid chromatography can be inefficient and expensive when dealing with kilogram quantities of material.

Accordingly, there remains a need to obtain the at least one compound of formula 1 in a more purified form than previously achieved. There also remains a need for new syntheses to minimize the use of chromatography for purification.

Disclosed herein are methods for producing tetracyclines, such as tigecycline, as generically illustrated in Scheme I below:

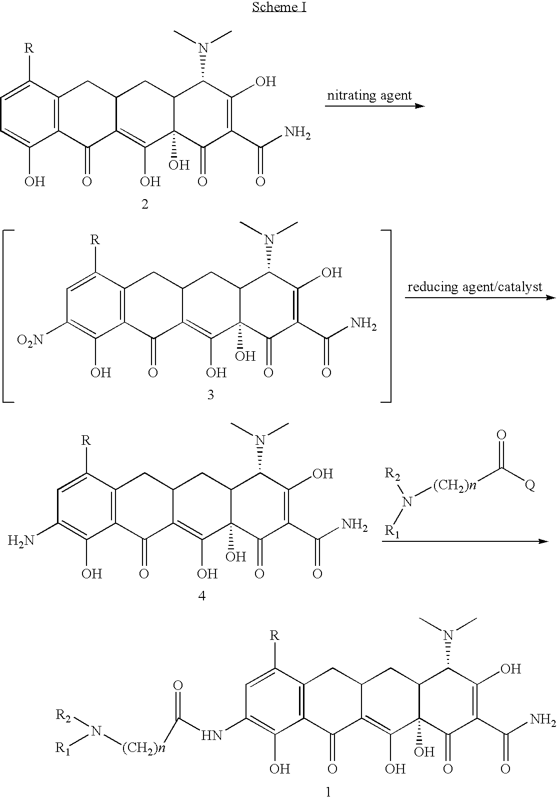

R₁ and R₂ are each independently chosen from hydrogen, straight and branched chain (C₁-C₆)alkyl, and cycloalkyl, or R₁ and R₂, together with N, form a heterocycle; and R is —NR₃R₄, where R₃ and R₄ are each independently chosen from hydrogen, and straight and branched chain (C₁-C₄)alkyl; and n ranges from 1-4.

The compound of formula 2 is also known as a minocycline or minocycline derivative. Reaction of the compound of formula 2 with at least one nitrating agent results in a —NO₂ substituent to form the compound of formula 3. The —NO₂ substituent in formula 3 can be subsequently reduced to an amino, such as by hydrogenation, to form the compound of formula 4. Finally, acylation of the compound of formula 4 generates the compound of formula 1.

Disclosed herein are methods for performing reactions to produce the compound formula 1, e.g., nitration, reduction, and acylation reactions. Also disclosed are methods for purifying the compound formula 1.

The methods disclosed herein can form the desired product while reducing the amount of at least one impurity present in the final product, such as epimer formation, the presence of starting reagents, and oxidation by-products. Such reduction in impurities can be achieved during at least one stage of the synthesis, i.e., during any one of the nitration, reduction, and acylation reactions. The methods disclosed herein can also facilitate large scale synthesis with suitable purities of the final products.

### DRAWINGS

FIG. 1 depicts an exemplary scheme for preparing tigecycline.

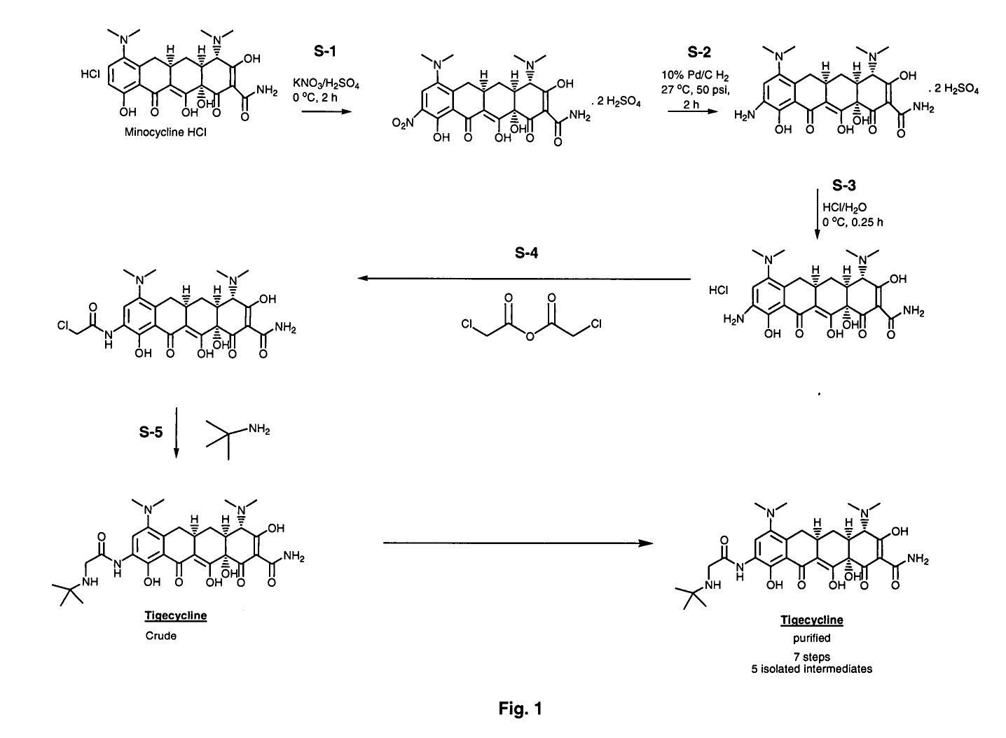

FIG. 2 depicts an exemplary scheme for preparing tigecycline.

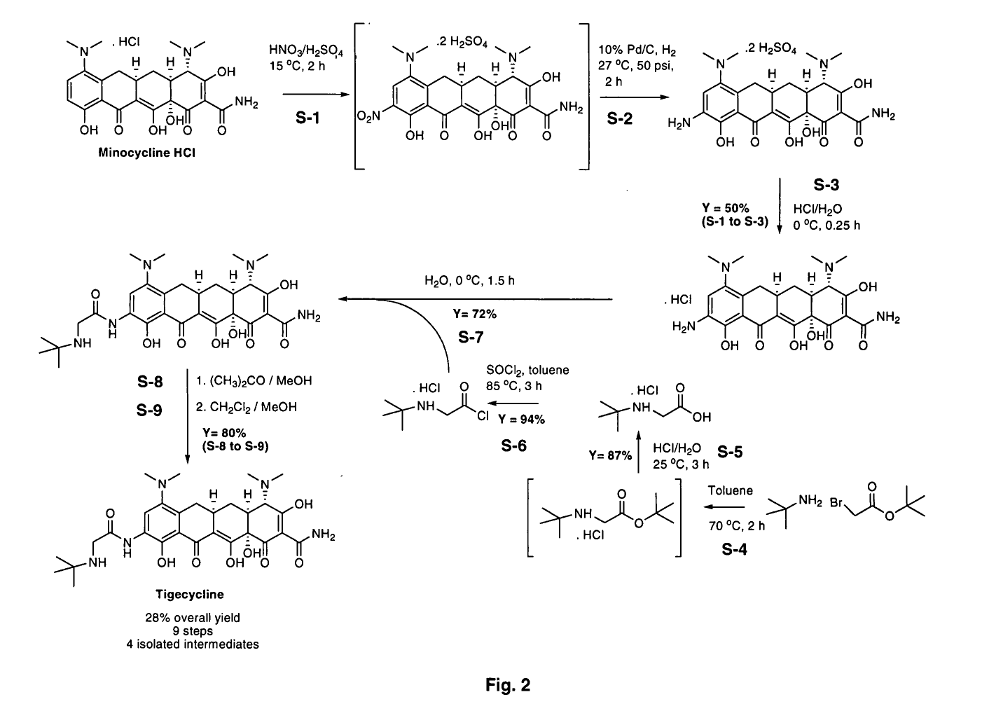

FIG. 3 depicts an exemplary scheme for preparing tigecycline.

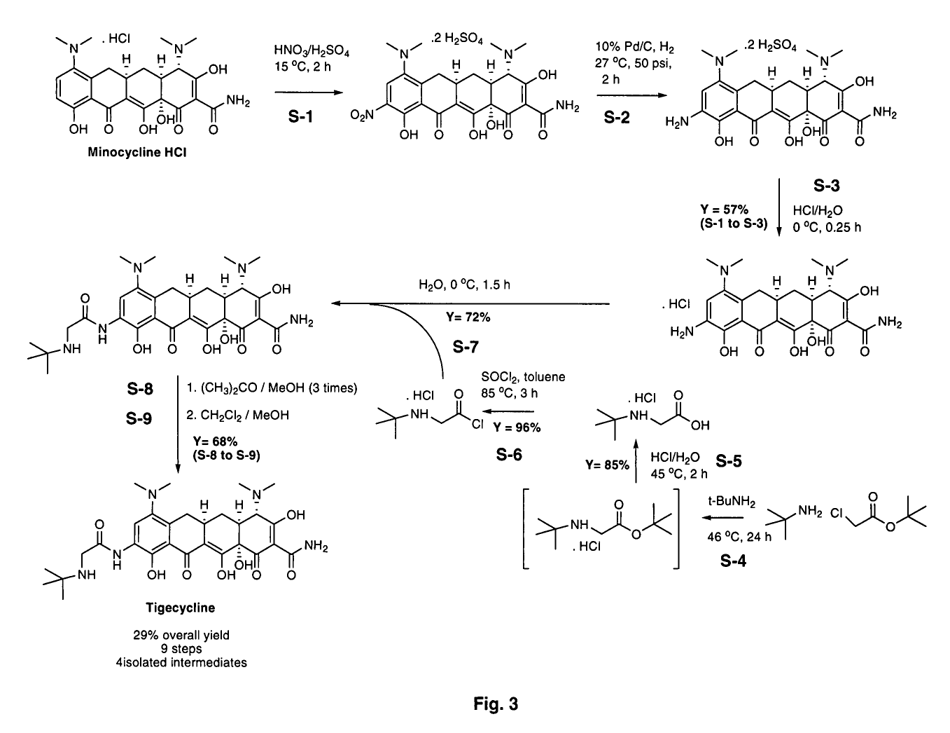

### DEFINITIONS

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

“Tigecycline” as used herein includes tigecycline in free base form and salt forms, such as any pharmaceutically acceptable salt, enantiomers, and epimers. Tigecycline, as used herein, may be formulated according to methods known in the art.

“Compound” as used herein refers to a neutral compound (e.g. a free base), and salt forms thereof (such as pharmaceutically acceptable salts). The compound can exist in anhydrous form, or as a hydrate, or as a solvate. The compound may be present as stereoisomers (e.g., enantiomers and diastereomers), and can be isolated as enantiomers, racemic mixtures, diastereomers, and mixtures thereof. The compound in solid form can exist in various crystalline and amorphous forms.

“Pharmaceutically acceptable” as used herein to refer to those compounds, materials, compositions, and/or dosage forms which are, within the scope of sound medical judgment, suitable for use in contact with the tissues of patients without excessive toxicity, irritation, allergic response, or other problem or complication commensurate with a reasonable risk/benefit ratio.

“Cycloalkyl” as used herein refers to a saturated carbocyclic ring system having 3 to 6 ring members.

“Heterocycle” as used herein refers to a monocyclic heterocycle group containing at least one nitrogen ring member and having 3 to 6 ring members in each ring wherein each ring is saturated and not otherwise substituted.

Nitration

One embodiment discloses a method of preparing at least one compound of formula 1,

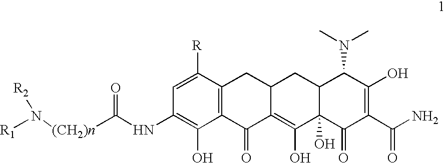

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4.

One embodiment discloses a nitration reaction where the product of the nitration is not isolated. Accordingly, in one embodiment, the method comprises:

(a) reacting at least one nitrating agent with at least one compound of formula 2,

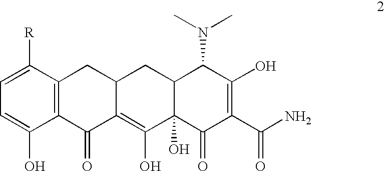

or a salt thereof, to produce a reaction mixture comprising an intermediate; and

(b) further reacting the intermediate to form the at least one compound of formula 1.

In one embodiment, the intermediate is not isolated from the reaction mixture.

The at least one compound of formula 2 can be provided as a free base or as a salt. In one embodiment, the at least one compound of formula 2 is a salt. “Salts” as used herein may be prepared in situ or separately by reacting a free base with a suitable acid. Exemplary salts include, but are not limited to, hydrochloride, hydrobromide, hydroiodide, phosphoric, nitric, sulfuric, acetic, benzoic, citric, cystein, fumaric, glycolic, maleic, succinic, tartaric, sulfate, and chlorobenzensulfonate salts. In another embodiment, the salt can be chosen from alkylsulfonic and arylsulfonic salts. In one embodiment, the at least one compound of formula 2 is provided as a hydrochloride salt, or as a sulfate salt.

“Nitrating agent” as used herein refers to a reagent that can add a —NO₂ substituent to a compound, or transform an existing substituent to an —NO₂ substituent. Exemplary nitrating reagents include nitric acid and nitrate salts, such as alkali metal salts, e.g., KNO₃. Where the nitrating agent is a nitric acid, the nitric acid can have a concentration of at least 80%, such as a concentration of 85%, 88%, 90%, 95%, 99%, or even 100%.

The nitrating agent can react with the at least one compound of formula 2 in any solvent deemed suitable by one of ordinary skill in the art. In one embodiment, the reaction is performed in the presence of sulfuric acid and/or sulfate salts. In one embodiment, the sulfuric acid used is concentrated sulfuric acid, e.g., a concentration of at least 50%, 60%, 70%, 80%, 85%, 90%, or at least 95%.

In one embodiment, the at least one nitrating agent is provided in a molar excess relative to the at least one compound of formula 2. Suitable molar excesses can be determined by one of ordinary skill in the art and can include, but are not limited to, values such as at least 1.05, e.g., a molar excess ranging from 1.05 to 1.75 equivalents, such as a molar excess ranging from 1.05 to 1.5, or from 1.05 to 1.25, or from 1.05 to 1.1 equivalents. In another embodiment, the molar excess is 1.05, 1.1, 1.2, 1.3, or 1.4 equivalents.

In one embodiment, the at least one nitrating agent is reacted with the at least one compound of formula 2 by adding the at least one nitrating agent over a period of time. One of ordinary skill in the art can determine a time period over which the total amount of nitrating agent is added to optimize the reaction conditions. For example, the addition of nitration reagent can be monitored by, for example, HPLC, to control the amount of the at least one nitrating agent used. In one embodiment, the total amount of the at least one nitrating agent is added over a period of time of at least 1 h, such as a period of time of at least 2 h, at least 3 h, at least 5 h, at least 10 h, at least 24 h, or a period of time ranging from 1 h to 1 week, ranging from 1 h to 48 h, ranging from 1 h to 24 h, or ranging from 1 h to 12 h.

The at least one nitrating agent can be added continuously.

In one embodiment, the nitrating agent can be reacted with the at least one compound of formula 2 at a temperature ranging from 0 to 25° C., such as a temperature ranging from 5 to 15° C., from 5 to 10° C., or from 10 to 15° C.

An “intermediate” as used herein refers to a compound that is formed as an intermediate product between the starting material and the final product. In one embodiment, the intermediate is a product of the nitration of at least one compound of formula 2. For example, the intermediate can be at least one compound of formula 3 or a salt thereof,

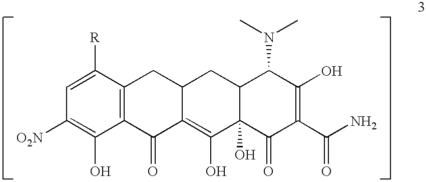

The intermediate can exist as a free base or as a salt, such as any of the salts disclosed herein. In one embodiment, the intermediate is a sulfate salt.

In one embodiment, the intermediate is not isolated from the reaction mixture. “Reaction mixture” as used herein refers to a solution or slurry comprising at least one product of a chemical reaction between reagents, as well as by-products, e.g., impurities (including compounds with undesired stereochemistries), solvents, and any remaining reagents, such as starting materials. In one embodiment, the intermediate is the product of the nitration and is present in the reaction mixture, which can also contain starting reagents (such as the nitrating agent and/or at least one compound of formula 2), by-products (such as the C₄-epimer of either formula 2 or formula 3). In one embodiment, the reaction mixture is a slurry, where a slurry can be a composition comprising at least one solid and at least one liquid (such as water, acid, or a solvent), e.g., a suspension or a dispersion of solids.

In one embodiment, the nitration reaction produces the intermediate while generating a low amount of the corresponding C₄-epimer. For example, where the intermediate is the at least one compound of formula 3, the nitration results in the formation of C₄-epimer of formula 3 in an amount less than 10%, as determined by high performance liquid chromatography (HPLC). In another embodiment, the C₄-epimer is present in an amount less than 5%, less than 3%, less than 2%, less than 1%, or less than 0.5%.

HPLC parameters for each step, i.e., nitration, reduction, and acylation, are provided in the Examples section.

In one embodiment, the nitration is performed such that the amount of starting material, e.g., the at least one compound of formula 2, is low. In one embodiment, the at least one compound of formula 2 is present in the nitration product in an amount less than 10%, as determined by HPLC, or less than 5%, less than 3%, less than 2%, less than 1%, or less than 0.5%.

In one embodiment, the nitration can be performed in a large scale. In one embodiment, “large scale” refers to the use of at least 1 gram of the compound according to formula 2, such as the use of at least 2 grams, at least 5 grams, at least 10 grams, at least 25 gram, at least 50 grams, at least 100 grams, at least 500 g, at least 1 kg, at least 5 kg, at least 10 kg, at least 25 kg, at least 50 kg, or at least 100 kg.

In one embodiment, the reducing forms at least one compound of formula 4,

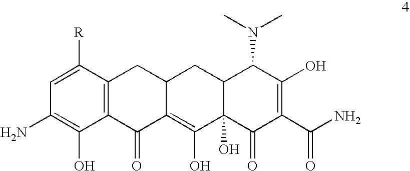

or a salt thereof.

In one embodiment, the further reacting in (b) comprises reducing the intermediate. In another embodiment, the method further comprises acylating the reduced intermediate.

Another embodiment disclosed herein is a method of preparing at least one compound of formula 1,

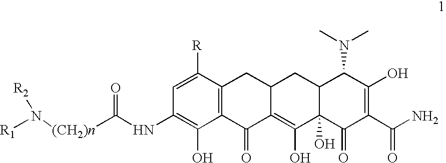

or a pharmaceutically acceptable salt thereof,

wherein R₁ is hydrogen, R₂ is t-butyl, R is —NR₃R₄ where R₃ is methyl and R₄ is methyl, and n is 1,

comprising:

(a) reacting at least one nitrating agent with at least one compound of formula 2,

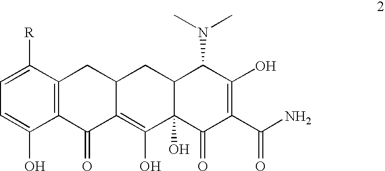

or a salt thereof, to produce a reaction mixture comprising an intermediate; and

(b) further reacting the intermediate to form the at least one compound of formula 1,

In one embodiment, the intermediate is not isolated from the reaction mixture.

In one embodiment, the at least one compound of formula 1 is tigecycline.

Another embodiment disclosed herein is a method of preparing at least one compound of formula 1,

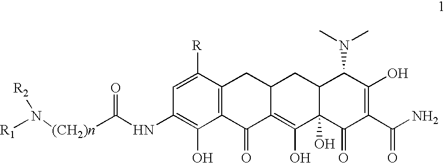

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4,

comprising:

(a) reacting at least one nitrating agent with at least one compound of formula 2,

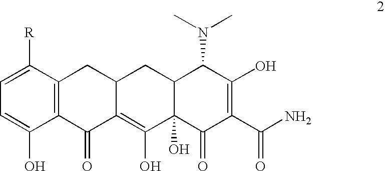

or a salt thereof, to produce a slurry; and

(b) further reacting the slurry to form the at least one compound of formula 1.

In one embodiment, R₁ is hydrogen, R₂ is t-butyl, R is —NR₃R₄ where R₃ is methyl and R₄ is methyl, and n is 1. In another embodiment, the at least one compound of formula 1 is tigecycline.

Another embodiment disclosed herein is a method of preparing at least one compound of formula 3 or a salt thereof,

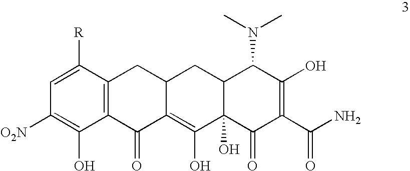

wherein R is —NR₃R₄, where R₃ and R₄ are each independently chosen from hydrogen, and straight and branched chain (C₁-C₄)alkyl,

comprising:

reacting at least one nitrating agent with at least one compound of formula 2 or a salt thereof,

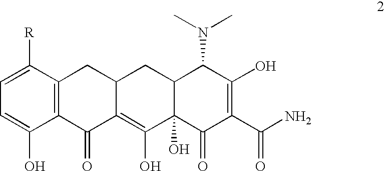

wherein the reacting is performed at a temperature ranging from 5 to 15° C.

Another embodiment disclosed herein is a method of preparing least one compound of formula 1,

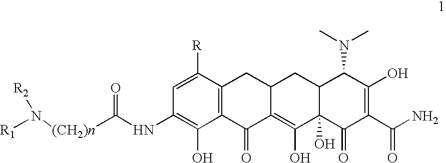

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4,

comprising:

(a) reacting at least one nitrating agent with at least one compound of formula 2 or a salt thereof to produce a reaction mixture comprising an intermediate; and

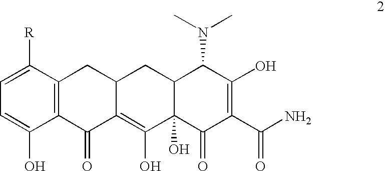

(b) further reacting the intermediate to form the at least one compound of formula 1

wherein the reacting in (a) is performed at a temperature ranging from 5 to 15° C.

In one embodiment, R₁ is hydrogen, R₂ is t-butyl, R₃ is methyl, R₄ is methyl, and n is 1.

Reduction

One embodiment discloses a method of preparing at least one compound of formula 4,

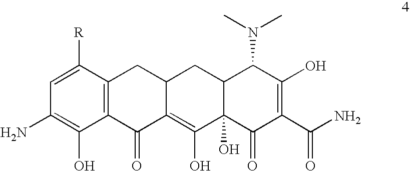

or a salt thereof,

wherein R=—NR₃R₄, where R₃ and R₄ are each independently chosen from hydrogen, and straight and branched chain (C₁-C₄)alkyl,

comprising:

combining at least one reducing agent with a reaction mixture, such as a reaction mixture slurry, comprising an intermediate prepared from a reaction between at least one nitrating agent and at least one compound of formula 2,

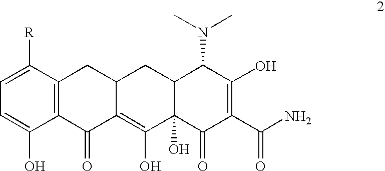

or a salt thereof.

In one embodiment, the method describes a “one-pot” process, where the nitration and reduction steps are performed without isolating the products of the nitration from the nitration reaction mixture.

In one embodiment, R₁ is hydrogen, R₂ is t-butyl, R₃ is methyl, R₄ is methyl, and n is 1.

“Reducing agent” as used herein refers to a chemical agent that adds hydrogen to a compound. In one embodiment, a reducing agent is hydrogen. The reduction can be performed under a hydrogen atmosphere at a suitable pressure as determined by one of ordinary skill in the art. In one embodiment, the hydrogen is provided at a pressure ranging from 1 to 75 psi, such as a pressure ranging from 1 to 50 psi, or a pressure ranging from 1 to 40 psi.

In another embodiment, the reducing agent is provided in the presence of at least one catalyst. Exemplary catalysts include, but are not limited to, rare earth metal oxides, Group VIII metal-containing catalysts, and salts of Group VIII metal-containing catalyst. An example of a Group VIII metal-containing catalyst is palladium, such as palladium on carbon.

Where the catalyst is palladium on carbon, in one embodiment, the catalyst is present in an amount ranging from 0.1 parts to 1 part, relative to the amount of the at least one compound of formula 2 present prior to the reaction with the at least one nitrating agent.

In one embodiment, the intermediate is at least one compound of formula 3. In one embodiment, in the compound of formula 3, R₁ is hydrogen, R₂ is t-butyl, R₃ is methyl, R₄ is methyl, and n is 1.

One of ordinary skill in the art can determine a suitable solvent for the reduction reaction. In one embodiment, prior to the combining, e.g., prior to the reduction, the reaction mixture is combined with a solvent comprising at least one (C₁-C₈) alcohol. The at least one (C₁-C₈) alcohol can be chosen, for example, from methanol and ethanol.

One of ordinary skill in the art can determine a suitable temperature for the reduction reaction. In one embodiment, the combining, e.g., the reduction, is performed at a temperature ranging from 0° C. to 50° C., such as a temperature ranging from 20° C. to 40° C., or a temperature ranging from 26° C. to 28° C.

In one embodiment, after the combining, e.g., after the reduction, the resulting reaction mixture is added to or combined with a solvent system comprising a (C₁-C₈) branched chain alcohol and a (C₁-C₈) hydrocarbon. In one embodiment, the (C₁-C₈) branched chain alcohol is isopropanol. In one embodiment, the (C₁-C₈) hydrocarbon is chosen from hexane, heptane, and octane.

In one embodiment, after the combining, e.g., after the reduction, the resulting reaction mixture is added to the solvent system at a temperature ranging from 0° C. to 50° C., such as a temperature ranging from 0° C. to 10° C.

In one embodiment, the method further comprises isolating the at least one compound of formula 4 as a solid, or as a solid composition. In one embodiment, the at least one compound of formula 4 is precipitated or isolated as a salt, such as any of the salts described herein.

In one embodiment, the solid composition comprises a C₄-epimer of formula 4 in an amount less than 10% as determined by high performance liquid chromatography. In another embodiment, the C₄-epimer is present in an amount less than 5%, less than 3%, less than 2%, less than 1%, or less than 0.5%.

In one embodiment, the solid composition comprises the at least one compound of formula 2 in an amount less than 2%, such as an amount less than 1%, or less than 0.5%, as determined by high performance liquid chromatography.

In one embodiment, the reduction can be performed in a large scale. In one embodiment, “large scale” refers to the use of at least 1 gram of the compound according to formula 2, such as the use of at least 2 grams, at least 5 grams, at least 10 grams, at least 25 gram, at least 50 grams, at least 100 grams, at least 500 g, at least 1 kg, at least 5 kg, at least 10 kg, at least 25 kg, at least 50 kg, or at least 100 kg.

Another embodiment disclosed herein is a method of preparing at least one compound of formula 1,

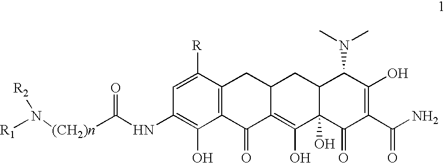

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4,

comprising:

(a) combining at least one reducing agent with a reaction mixture, such as a reaction mixture slurry, comprising an intermediate prepared from a reaction between at least one nitrating agent and at least one compound of formula 2,

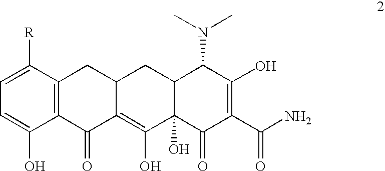

or a salt thereof, to form a second intermediate; and

(b) further reacting the second intermediate in the reaction mixture to prepare the at least one compound of formula 1.

In one embodiment, R₁ is hydrogen, R₂ is t-butyl, R₃ is methyl, R₄ is methyl, and n is 1.

In one embodiment, the intermediate is at least one compound of formula 3 or salt thereof, and the second intermediate is at least one compound of formula 4,

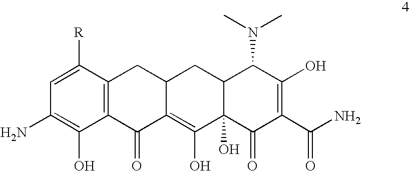

or a salt thereof.

In one embodiment, the further reacting in (b) comprises acylating the second intermediate. In one embodiment, prior to the acylating, the second intermediate can be precipitated or isolated as a salt.

Another embodiment disclosed herein is a method of preparing at least one compound of formula 4 or a salt thereof,

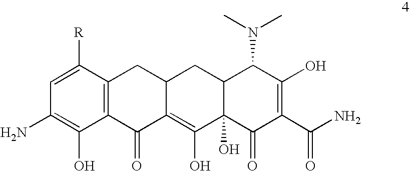

wherein R=—NR₃R₄, where R₃ and R₄ are each independently chosen from hydrogen, and straight and branched chain (C₁-C₄)alkyl,

comprising:

reducing an intermediate of formula 3 or a salt thereof,

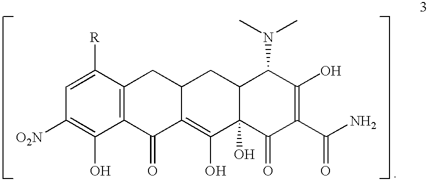

In one embodiment, the intermediate of formula 3 may be present in a reaction mixture slurry.

In one embodiment, the reducing comprises combining at least one reducing agent with the reaction mixture.

Another embodiment disclosed herein is a method of preparing at least one compound of formula 1,

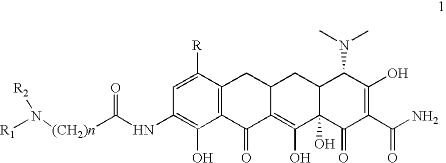

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4,

comprising:

(a) reacting at least one nitrating agent with at least one compound of formula 2 or a salt thereof to prepare a reaction mixture,

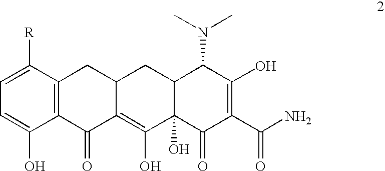

(b) without isolating or precipitating any solids from the reaction mixture, combining at least one reducing agent with the reaction mixture to prepare an intermediate; and

(c) preparing the at least one compound of formula 1 from the intermediate.

Another embodiment disclosed herein is method of preparing at least one compound of formula 1,

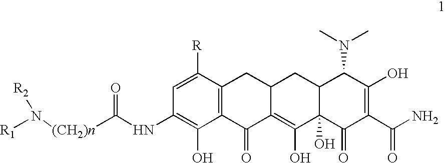

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4,

comprising:

(a) combining at least one Group VIII metal-containing catalyst in the presence of hydrogen with a reaction mixture, such as a reaction mixture slurry, prepared from a reaction between at least one nitrating agent and at least one compound of formula 2 or a salt thereof,

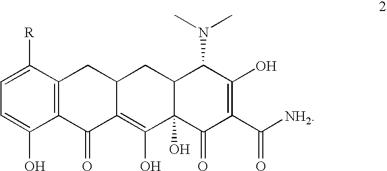

In one embodiment, the at least one Group VIII metal-containing catalyst is present in an amount ranging from 0.1 parts to 1 part relative to the amount of the at least one compound of formula 2 present prior to the reaction with the at least one nitrating agent.

Another embodiment disclosed herein is a composition comprising:

at least one compound of formula 4,

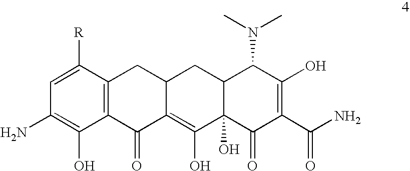

or a salt thereof,

wherein R is —NR₃R₄, where R₃ and R₄ are each independently chosen from hydrogen, and straight and branched chain (C₁-C₄)alkyl,

wherein a C₄-epimer of formula 4 is present in an amount less than 10%, as determined by high performance liquid chromatography.

In one embodiment, R₁ is hydrogen, R₂ is t-butyl, R₃ is methyl, R₄ is methyl, and n is 1.

Acylation

One embodiment of the present disclosure provides a method for preparing at least one compound of Formula 1:

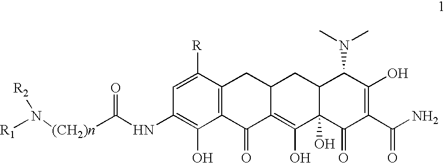

or a pharmaceutically acceptable salt thereof,

wherein R₁ and R₂ are each independently chosen from hydrogen, straight and branched chain (C₁-C₆)alkyl, and cycloalkyl, such as (C₃-C₆)cycloalkyl, or R₁ and R₂, together with N, form a heterocycle, such as a 5-membered ring; R is —NR₃R₄, where R₃ and R₄ are each independently chosen from hydrogen, and straight and branched (C₁-C₄)alkyl; and n ranges from 1-4,

comprising reacting at least one compound of Formula 4:

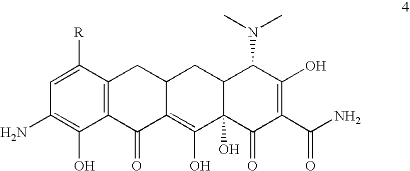

or a salt thereof,

with at least one aminoacyl compound in a reaction medium. In one embodiment, the reaction medium may be chosen from an aqueous medium, and at least one basic solvent in the absence of a reagent base.

In one embodiment, the method for preparing a compound of formula I is a method for preparing tigecycline:

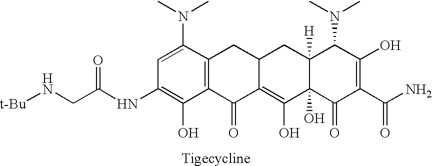

or a pharmaceutically acceptable salt thereof.

In one embodiment, variable n is 1, R₁ is hydrogen, R₂ is t-butyl, and R₃ and R₄ are each methyl. In another embodiment, variable n is 1, R₁ and R₂, together with N, forms a pyrrolidinyl group, and R₃ and R₄ are each methyl. The salt of the at least one compound of Formula 4 may be a halogenated salt, such as a hydrochloride salt.

The reaction medium may be a solvent chosen from a polar aprotic solvent or mixture of solvents thereof. In one embodiment, the polar aprotic solvent is chosen from acetonitrile, 1,2-dimethoxyethane, dimethylacetamide, dimethylformamide, hexamethylphosphoramide, N,N′-dimethylethyleneurea, N,N′-dimethylpropyleneurea, methylene chloride, N-methylpyrrolidinone, tetrahydrofuran, and mixtures thereof. In another embodiment, the polar aprotic solvent is chosen from acetonitrile, dimethylformamide, N,N′-dimethylpropyleneurea, N-methylpyrrolidinone, tetrahydrofuran, and mixtures thereof. The at least one basic solvent may be a mixture of acetonitrile and N,N′-dimethylpropyleneurea. In another embodiment, the at least one basic solvent may be a mixture of water and N,N′-dimethylpropyleneurea. In a further embodiment, the at least one basic solvent is N,N′-dimethylpropyleneurea.

The reaction medium may be an aqueous medium. In a further embodiment, the at least one basic solvent in the absence of a base is water in the absence of a base. In another embodiment, the reaction medium may be at least one basic solvent in the absence of a reagent base. A basic solvent is a solvent capable of accepting, either partially or fully, a proton. A reagent base refers to a base that is added at the start of the reaction, either concurrently or sequentially with the at least one compound of Formula 4 and the at least one aminoacyl compound and is capable of accepting, either partially or fully, a proton. A reagent base also refers to a base that is added during the reaction.

The at least one aminoacyl compound may be chosen from aminoacyl halides, aminoacyl anhydrides, and mixed aminoacyl anhydrides. In one embodiment, the aminoacyl compound is at least one aminoacyl halide of Formula 6:

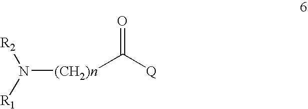

or a salt thereof,

wherein R₁ and R₂ are each independently chosen from hydrogen, straight and branched chain (C₁-C₆)alkyl, and cycloalkyl, or R₁ and R₂, together with N, form a heterocycle; n ranges from 1-4; and wherein Q is a halogen chosen from fluoride, bromide, chloride, and iodide.

In a further embodiment, Q is chloride. The salt of the compound of Formula 6 may be chosen from a halogenated salt. Halogenated salt refers to any salt formed from interaction with a halogen anion, such as a hydrochloride salt, a hydrobromide salt, and a hydroiodic salt. In one embodiment, the halogenated salt is a hydrochloride salt.

The at least one aminoacyl halide of Formula 6 may be obtained by a method comprising:

A) reacting at least one ester of Formula 7:

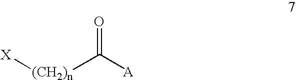

or a salt thereof,

with at least one amine, R₁R₂NH, to prepare at least one carboxylic acid,

wherein R₁ and R₂ are each independently chosen from hydrogen, straight and branched chain (C₁-C₆)alkyl, and cycloalkyl, or R₁ and R₂, together with N, form a heterocycle; X is a halogen chosen from bromide, chloride, fluoride and iodide; A is —OR₆, where R₆ is chosen from straight or branched (C₁-C₆)alkyl and arylalkyl, such as aryl(C₁-C₆)alkyl, e.g., where aryl is phenyl; n ranges from 1-4; and

B) reacting the at least one carboxylic acid with at least one chlorinating agent to give at least one aminoacyl compound of Formula 6 or a salt thereof.

In one embodiment, R₁ and R₆ may each be t-butyl. In another embodiment, R₁ and R₂, together with N, may form a heterocycle, such as pyrrolidine, and R₆ may be arylalkyl, such as benzyl. In another embodiment, n is one. In a further embodiment, X is bromide.

In another embodiment, the at least one ester of Formula 7 is a hydrochloride salt. An excess of amine R₁R₂NH compared to the ester of Formula 7 may be present in the reaction to prepare at least one carboxylic acid. In one embodiment, the at least one chlorinating agent may be thionyl chloride. In another embodiment, the reaction of the at least one carboxylic acid with at least one chlorinating agent includes addition of a catalytic amount of dimethylformamide. An excess of chlorinating agent relative to the at least one carboxylic acid may be present in the reaction to give at least one aminoacyl compound of Formula 6. When R₆ is arylalkyl, the arylalkyl of the at least one compound of Formula 7 may be cleaved by hydrogenation after reaction with the at least one amine to give the at least one carboxylic acid.

The reaction of the at least one carboxylic acid with a chlorinating agent may be performed at a temperature ranging from 55° C. to 85° C., such as from 80° C. to 85° C., and further such as 55° C. In one embodiment, an additional amount of chlorinating agent may be added to the reaction to effect completion, such as attaining a level of less than 4% carboxylic acid. Following reacting the at least one carboxylic acid with at least one chlorinating agent, the resulting suspension may be filtered to remove salts, such as t-butylamine hydrochloride salts. The aminoacyl halide of Formula 6 may be isolated as HCl salt or treated with an inorganic acid, such as hydrochloric acid, to prepare an aminoacyl halide salt.

In another embodiment, the at least one aminoacyl halide of Formula 6 is obtained by a method comprising:

reacting at least one carboxylic acid of Formula 8:

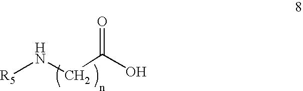

or a salt thereof,

wherein R₅ is chosen from straight or branched (C₁-C₆)alkyl, and n ranges from 1 to 4, and

with at least one chlorinating agent to give at least one aminoacyl halide of Formula 6 or a salt thereof.

In another embodiment, the at least one carboxylic acid of Formula 8 is a halogenated salt, such as a hydrochloride salt. The time period for reacting at least one compound of Formula 8 with at least one chlorinating agent may range from 1 to 50 hours, such as from 2 to 45 hours, and further such as 1 to 3 hours. The at least one carboxylic acid of Formula 8 may have a particle size of less than 150 microns, such as less than 110 microns, and further such as ranging from 50 to 100 microns. A compound of Formula 8 having a given particle size may be attained by milling the compound.

Reacting at least one compound of Formula 4 with the at least one aminoacyl compound may be conducted at a temperature ranging from 0° C. to 30° C., such as from 20° C. to 25° C., such as from 10° C. to 17° C., such as from 0° C. to 6° C., and further such as from 2° C. to 8° C. The time period for reaction may range from 1 hour to 24 hours, such as from 0.5 hours to 4 hours, and further such as from 2 hours to 8 hours. An excess of aminoacyl compound relative to the amount of a compound of Formula 4 may be used in the reaction. In one embodiment, the excess may be 3 equivalents of aminoacyl compound to 1 equivalent of the at least one compound of Formula 4. In another embodiment, the ratio of aqueous medium to the at least one compound of Formula 4 may be 6:1 w/w or 5:1 volumes. In one embodiment, the aminoacyl compound is added to or combined with a solution of the at least one compound of Formula 4 in an aqueous medium.

In one embodiment, where the reaction medium is an aqueous medium, the pH of the aqueous medium may be adjusted to a pH ranging from 4 to 9, such as from 5 to 7.5, such as from 6.3 to 6.7, such as from 7.0 to 7.5, further such as 6.5, and still further such as 7.2. Water may be added prior to adjusting the pH. Adjusting the pH may involve addition of a base, including but not limited to ammonium hydroxide. The concentration of ammonium hydroxide may range from 25% to 30%. In another embodiment, an acid, such as hydrochloric acid, may be used to adjust the pH. The reaction medium during pH adjustment may be at a temperature ranging from −5° C. to 25° C., such as from 5° C. to 8° C., and further such as from 0° C. to 5° C.

Following adjustment of the pH, at least one organic solvent or mixture of solvents may be added to the aqueous medium. In one embodiment, the at least one organic mixture of solvents may comprise methanol and methylene chloride. The concentration of methanol may range from 5% to 30%, including but not limited to 20% and 30%. In another embodiment, the at least one organic solvent or mixture of solvents comprises tetrahydrofuran. The temperature of the mixture may range from 15° C. to 25° C.

In one embodiment, the aqueous medium may be extracted with a mixture of at least one polar protic solvent and at least one polar aprotic solvent. In one embodiment, the at least one polar aprotic solvent comprises methylene chloride and the at least one polar protic solvent comprises methanol. In another embodiment, the aqueous medium is extracted with at least one polar aprotic solvent, such as methylene chloride. The extraction may be conducted at a temperature ranging from −5° C. to 25° C., further such as from 0° C. to 5° C. In a further embodiment, the pH of the aqueous medium is adjusted to a range from 7.0 to 7.5, such as 7.2, after each extraction. The extraction process may be repeated, for example, up to 10 times.

In one embodiment, the combined organic extracts may be treated with a drying agent, such as sodium sulfate. The organic extracts may also be treated with charcoal, such as Norit CA-1. The solids are removed by filtration to give a solution. In one embodiment, the solution may be concentrated to afford the compound of Formula 1.

The compound of Formula 1 obtained from the reaction may be crystallized in at least one organic solvent or mixture of solvents. In one embodiment, the organic mixture of solvents comprises methanol and methylene chloride. Crystallization may, for example, occur at a temperature ranging from −15° C. to 155° C., such as from 0° C. to 15° C., and further such as from 2° C. to 5° C.

In another embodiment, following extraction, the resulting organic mixture of at least one polar protic solvent and at least one polar aprotic solvent may be concentrated to give a slurry and filtered to give the at least one compound of Formula 1. Concentration and filtration may, for example, occur at 0° C. to 5° C.

A method for preparing a compound of Formula 1 may be performed using greater than 5 grams of the amine of Formula 4, such as greater than 10 grams, such as greater than 50 grams, such as greater than 100 grams, such as greater than 500 grams, such as greater than 1 kilograms, and further such as greater than 10 kilograms.

One embodiment discloses a compound prepared by any of the methods described herein, including but not limited to a compound of Formula 1, a compound of Formula 4, a compound of Formula 6, a compound of Formula 7, a compound of Formula 8, and salts thereof. Another embodiment includes a composition comprising a compound prepared by any of the methods described herein. The composition may further comprise a pharmaceutically acceptable carrier.

In one embodiment, the composition may comprise at least one compound of Formula 1:

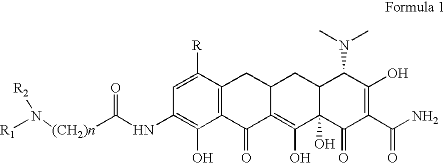

or a pharmaceutically acceptable salt thereof,

wherein n is 1, R₁ and R₂, together with N, forms a t-butyl group, and R₃ and R₄ are each methyl. In another embodiment, the composition may comprise at least one compound of formula 1:

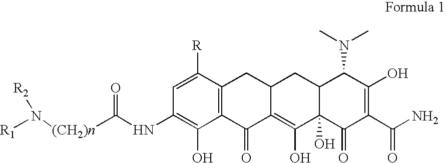

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched (C₁-C₄)alkyl; and n ranges from 1-4, and

less than 0.5% of the C-4 epimer of the at least one compound of formula 1 or a pharmaceutically acceptable salt thereof.

In a further embodiment, the composition may comprise Tigecycline:

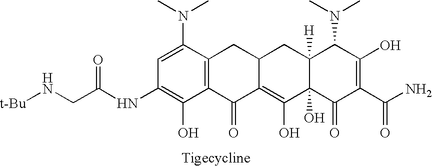

or a pharmaceutically acceptable salt thereof, and

less than 0.5% of the C-4 epimer of Tigecycline or a pharmaceutically acceptable salt thereof.

In one embodiment, the compound of Formula 1 prepared by any of the methods described herein contains less than 10.0% impurities as determined by high performance liquid chromatography, such as less than 5% impurities, such as less than 2% impurities, and further such as 1-1.4% impurities. In a further embodiment, the compound of Formula 1 contains a C₄-epimer in an amount less than 1.0% as determined by high performance liquid chromatography, such as less than 0.5% C₄-epimer, and further such as less than 0.2% C₄-epimer. In one embodiment, the compound of formula 1 contains less that 1% minocycline as determined by high performance liquid chromatography, such as less than 0.6% minocycline. In another embodiment, the compound of formula 1 contains less than 5% dichloromethane, such as less than 2-3% dichloromethane.

One embodiment of the disclosure includes a method for preparing at least one compound of Formula 1:

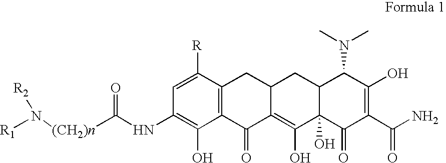

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched (C₁-C₄)alkyl; and n ranges from 1-4,

comprising:

A) reacting at least one nitrating agent with at least one compound of Formula 2:

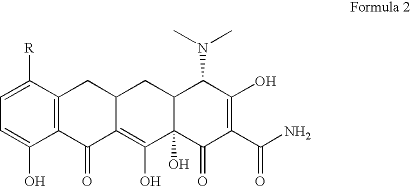

or a salt thereof,

to prepare a reaction mixture slurry comprising at least one compound of Formula 3:

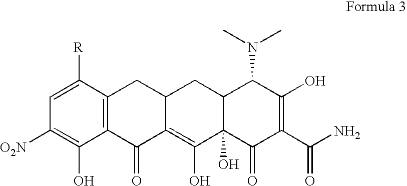

or a salt thereof,

B) combining at least one reducing agent with the reaction mixture slurry to prepare at least one compound of Formula 4,

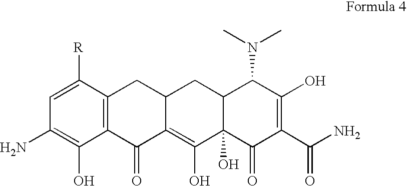

or a salt thereof, and

C) reacting the at least one compound of Formula 4 with at least one aminoacyl compound in a reaction medium chosen from an aqueous medium, and at least one basic solvent in the absence of a reagent base.

The compound of formula I prepared by this method may be tigecyline.

Another embodiment of the present disclosure includes a method for preparing at least one compound of Formula 1:

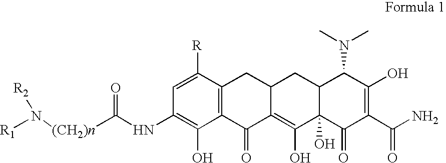

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched (C₁-C₄)alkyl; and n ranges from 1-4,

comprising:

A) combining at least one reducing agent with a reaction mixture slurry comprising at least one compound of Formula 3:

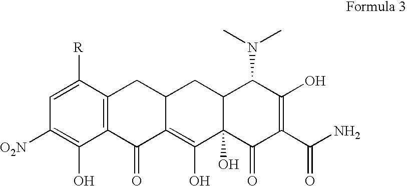

or a salt thereof,

to prepare at least one compound of Formula 4:

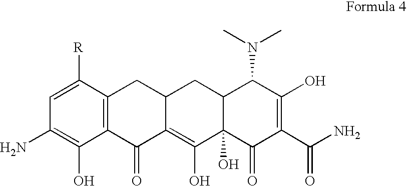

or a salt thereof, and

B) reacting the at least one compound of Formula 4 with at least one aminoacyl compound in a reaction medium chosen from an aqueous medium, and at least one basic solvent in the absence of a reagent base.

In another embodiment, the compound of formula I prepared by the above method may be tigecyline.

Purification

One embodiment of the present disclosure provides a method for purifying at least one compound of Formula 1:

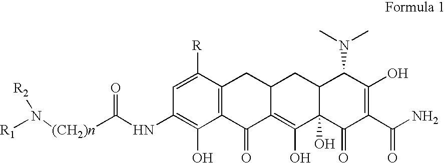

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched (C₁-C₄)alkyl; and n ranges from 1-4,

comprising:

A) combining the at least one compound of Formula 1 with at least one polar aprotic solvent and at least one polar protic solvent to give a first mixture,

B) mixing the first mixture for at least one period of time such as from 15 minutes to 2 hours at a temperature ranging from 0° C. to 40° C., and

C) obtaining the at least one compound of Formula 1.

As used herein, the term “obtaining” refers to isolating a compound at a useful level of purity, including but not limited to levels of purity greater than 90%, 95%, 96%, 97%, 98%, and 99%. The level of purity may be determined by high pressure liquid chromoatography.

In one embodiment, the method for purifying at least one compound of Formula 1 involves the steps of:

A) combining the at least one compound of Formula 1 with at least one polar aprotic solvent and at least one polar protic solvent to give a first mixture,

B) mixing the first mixture for a period of time at a temperature ranging from 30° C. to 40° C.,

C) cooling the first mixture to a temperature ranging from 15° C. to 25° C. and

allowing the mixture to stand without mixing for a second period of time,

D) cooling the first mixture to a temperature ranging from 0° C. to 6° C. and

allowing the mixture to stand without mixing for a third period of time, and

E) obtaining the at least one compound of Formula 1.

In one embodiment, the method may include at least one compound of Formula 1 where n is 1, R₁ is hydrogen, R₂ is t-butyl, and R₃ and R₄ are each methyl. Another embodiment includes at least one compound of Formula 1, where n is 1, R₁ and R₂, together with N, forms a pyrrolidinyl group, and R₃ and R₄ are each methyl. The at least one compound of Formula 1 that is combined with the at least one polar aprotic solvent and the at least one polar protic solvent may be provided in a form chosen from a solid, a slurry, a suspension, and a solution.

In one embodiment, the at least one polar aprotic solvent may chosen from acetone, 1,2-dichloroethane, methyl acetate, methyl ethyl ketone, methyl isobutyl ketone, methylene chloride, and ethyl acetate. In a further embodiment, the at least one polar aprotic solvent may be chosen from acetone and methylene chloride. In another embodiment, the at least one polar protic solvent may be chosen from methanol, ethanol, isopropanol, and t-butanol. In a further embodiment, the at least one polar protic solvent may be methanol.

The combination of the at least one polar aprotic solvent and at least one polar protic solvent may include acetone and methanol. Another embodiment provides a combination of the at least one polar aprotic solvent, methylene chloride, and the at least one polar protic solvent, methanol. In a further embodiment, the combination of the at least one polar aprotic solvent and at least one polar protic solvent may include methyl acetate and methanol. The compound of Formula 1 may, for example, be combined with equal volumes of the at least one polar aprotic solvent and the at least one polar protic solvent.

In one embodiment, the first mixture may, for example, be mixed for a first period of time ranging from 30 minutes to 2 hours where the temperature ranges from 15° C. to 25° C., then for a second period of time ranging from 30 minutes to 2 hours, where the temperature ranges from 0° C. to 2° C. In one embodiment, the first period of time and the second period of time are each 1 hour. In another embodiment, the method may comprise mixing the first mixture for at least one period of time ranging from 30 minutes to 2 hours at a temperature ranging from 15° C. to 25° C., then filtering the first mixture to obtain a solid. The method may further comprise combining the solid with at least one polar aprotic solvent and at least one polar protic solvent, such as at equal volumes, for a first period of time ranging from 30 minutes to 2 hours at a temperature ranging from 15° C. to 25° C., and filtering to obtain a second solid. In a further embodiment, these combining and filtering steps may be repeated two to fifteen times.

The method for purifying a compound of Formula 1 may further comprise obtaining a solid from the first mixture, and combining the solid with at least one polar protic solvent and at least one polar aprotic solvent to obtain a second mixture. The second mixture may, for example, comprise methanol and methylene chloride in a ratio by volume ranging from 1:5 to 1:15 methanol:methylene chloride. In one embodiment, the second mixture may be mixed at a temperature ranging from 30° C. to 36° C. and then filtered to obtain a solution. In a further embodiment, the concentration of the polar protic solvent in the solution may be reduced to a level below 5%, and the solution may be mixed, for example, at a temperature ranging from 0° C. to 6° C., for a time period, for example, ranging from 30 minutes to 2 hours prior to filtering.

In one embodiment, mixing the first mixture may occur during a period of time ranging from 10 to 20 minutes, such as 15 minutes. In one embodiment, cooling the first mixture to a temperature ranging from 15° C. to 25° C. and allowing the mixture to stand without mixing may occur during a second period of time ranging from 30 minutes to 3 hours, such as from 1 hour to 2 hours. The first mixture may be further cooled to a temperature ranging from 0° C. to 6° C. and allowed to stand without mixing for a third period of time ranging from 30 minutes to 2 hours, such as 1 hour.

Obtaining the compound of Formula 1 may include filtering any mixture described herein through at least one filter selected from pyrogen reducing filters and clarifying filters.

As disclosed herein, mixing may be carried out by using a mechanical mixing device, for instance, a stirrer or agitator. Mixing may also be effected by solubility of the compound having Formula 1 in the solvent system. Increasing the temperature may increase solubility.

In one embodiment, when at least one compound of Formula 1 is to be combined with at least one polar aprotic solvent and at least one polar protic solvent, the at least one compound of Formula 1 may be used in the form of a pharmaceutically acceptable salt thereof. Where at least one compound of Formula 1 is obtained as the product of the method of the invention, the at least one compound of Formula 1 may be recovered in the form of a pharmaceutically acceptable salt thereof.

In another embodiment, where a compound of Formula 1 is obtained by the method according to the invention, the compound may be converted into a pharmaceutically acceptable salt thereof by addition of an acid.

In one embodiment, the at least one compound of Formula 1 may be [4S-(4α,12aα)]-4,7-Bis(dimethylamino)-9-[[(t-butylamino)acetyl]amino]-1,4,4a,5,5a,6,11,12a-octahydro-3,10,12,12a-tetrahydroxy-1,11-dioxo-2-naphthacene-carboxamide, such as pharmaceutically acceptable salts such as HCl salts. In another embodiment, the at least one compound of Formula 1 may be [4S-(4α,12aα)]-4,7-Bis(dimethylamino)-9-[[(pyrrolidinyl)acetyl]amino]-1,4,4a,5,5a,6,11,12a-octahydro-3,10,12,12a-tetrahydroxy-1,11-dioxo-2-naphthacene-carboxamide, such as pharmaceutically acceptable salts such as HCl salts.

A method for purifying at least one compound of Formula 1 may be a method for purifying tigecycline, comprising:

A) combining tigecycline with at least one polar aprotic solvent and at least one polar protic solvent to give a first mixture,

B) mixing the first mixture for at least one period of time, for example, ranging from 15 minutes to 2 hours and at a temperature ranging from 0° C. to 40° C., and

C) obtaining tigecycline.

The tigecycline that is combined with at least one polar aprotic solvent and at least one polar protic solvent may be provided in a form chosen from a solid, a slurry, a suspension, and a solution. In one embodiment, the tigecycline obtained from the method may contain less than 1% of the C-4 epimer of tigecycline or a pharmaceutically acceptable salt thereof as determine by high pressure liquid chromatography (HPLC).

The at least one compound of Formula 1 obtained from the method may contain less than 3.0% impurities as determined by HPLC, such as less than 1.0% impurities, such as less than 0.7% impurities. In another embodiment, the at least one compound of Formula 1 may contain less than 2% of the C-4 epimer of the compound of formula 1 or a pharmaceutically acceptable salt thereof, as determined by HPLC, such as less than 1% of the C-4 epimer, such as less than 0.5% of the C-4 epimer.

The method may be performed on greater than 5 grams of the at least one compound of Formula 1, such as greater than 50 grams, such as greater than 100 grams, such as greater than 500 grams, such as greater than 1 kilogram, and further such as greater than 10 kilograms.

One embodiment discloses a compound prepared by any of the methods described herein, including but not limited to a compound of Formula 1 and tigecycline. Another embodiment includes a composition comprising a compound prepared by any of the methods described herein. The composition may further comprise a pharmaceutically acceptable carrier.

In one embodiment, the composition may comprise at least one compound of Formula 1:

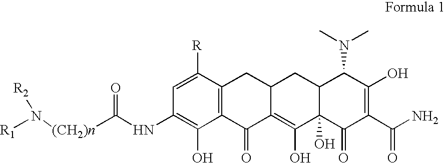

or a pharmaceutically acceptable salt thereof,

wherein n is 1, R₁ is hydrogen, R₂ is t-butyl, and R₃ and R₄ are each methyl.

One embodiment of the disclosure includes a method for preparing at least one compound of Formula 1:

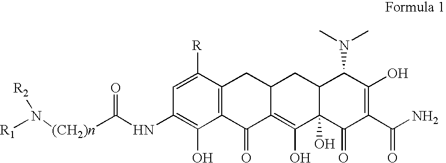

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched (C₁-C₄)alkyl; and n ranges from 1-4,

comprising:

A) reacting at least one nitrating agent with at least one compound of Formula 2:

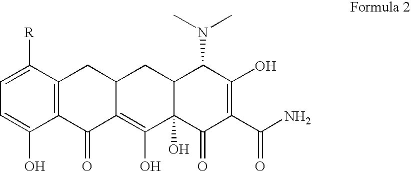

or a salt thereof,

to prepare a reaction mixture, such as a reaction mixture slurry, comprising an intermediate, such as at least one compound of Formula 3:

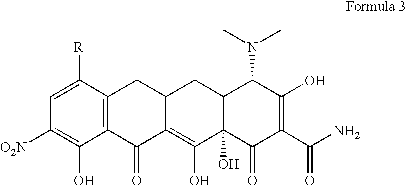

or a salt thereof,

B) combining at least one reducing agent with the reaction mixture slurry to prepare a second intermediate, such as at least one compound of Formula 4,

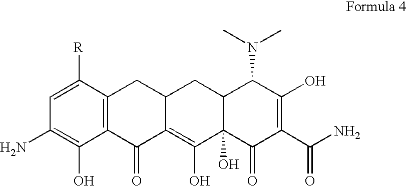

or a salt thereof,

C) reacting the second intermediate with at least one aminoacyl compound in a reaction medium to obtain at least one compound of formula 1. In one embodiment, the reaction medium is chosen from an aqueous medium, and at least one basic solvent in the absence of a reagent base. Additional steps may include, for example at lest one of:

D) combining the at least one compound of Formula 1 with at least one polar aprotic solvent and at least one polar protic solvent to give a first mixture,

E) mixing the first mixture for at least one period of time, such as ranging from 15 minutes to 2 hours, at a temperature, such as ranging from 0° C. to 40° C., and

F) obtaining at least one compound of Formula 1. In one embodiment, any of the intermediates of the methods disclosed may be isolated or precipitated out. In another embodiment, two or more steps of any of the methods disclosed are “one-pot” procedures.

Another embodiment of the disclosure includes a method for preparing at least one compound of Formula 1:

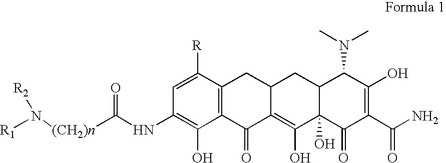

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched (C₁-C₄)alkyl; and n ranges from 1-4,

comprising:

A) combining at least one reducing agent with a reaction mixture, such as a reaction mixture slurry, comprising at least one compound of Formula 3:

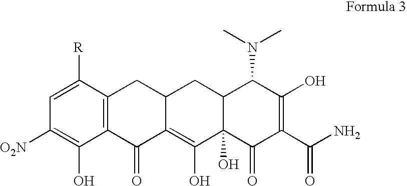

or a salt thereof, to prepare at least one intermediate, such as a compound of Formula 4,

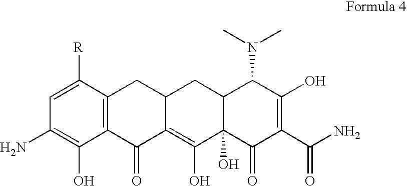

or a salt thereof,

B) reacting the intermediate with at least one aminoacyl compound in a reaction medium chosen from an aqueous medium to obtain the compound of Formula 1. In one embodiment, the reaction medium may be chosen from at least one basic solvent in the absence of a reagent base. Additional steps may include, for example, at least one of:

C) combining the at least one compound of Formula 1 with at least one polar aprotic solvent and at least one polar protic solvent to give a first mixture,

D) mixing the first mixture for at least one period of time, such as ranging from 15 minutes to 2 hours, at a temperature, such as ranging from 0° C. to 40° C., and

E) obtaining at least one compound of Formula 1.

A further embodiment of the disclosure includes a method for preparing at least one compound of Formula 1:

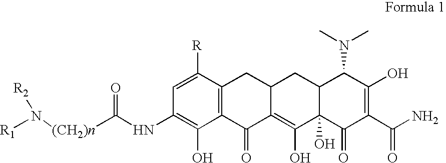

or a pharmaceutically acceptable salt thereof,

wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched (C₁-C₄)alkyl; and n ranges from 1-4, comprising:

A) reacting at least one compound of Formula 4:

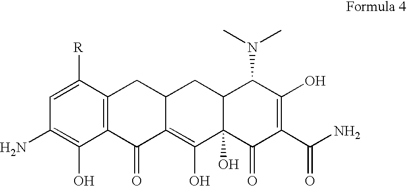

or a salt thereof,

with at least one aminoacyl compound in a reaction medium, for example, chosen from an aqueous medium, and at least one basic solvent in the absence of a reagent base to obtain the compound of Formula 1. Additional steps may include at least one of:

B) combining the at least one compound of Formula 1 with at least one polar aprotic solvent and at least one polar protic solvent to give a first mixture,

C) mixing the first mixture for at least one period of time, such as ranging from 15 minutes to 2 hours, at a temperature, such as ranging from 0° C. to 40° C., and

D) obtaining at least one compound of Formula 1.

Any of these methods disclosed for preparing a compound of Formula 1 may be a method for preparing a compound of Formula 1, where n is 1, R₁ is hydrogen, R₂ is t-butyl, and R₃ and R₄ are each methyl.

Pharmaceutical Compositions

“Pharmaceutical composition” as used herein refers to a medicinal composition. The pharmaceutical composition may contain at least one pharmaceutically acceptable carrier.

“Pharmaceutically acceptable excipient” as used herein refers to pharmaceutical carriers or vehicles suitable for administration of the compounds provided herein including any such carriers known to those skilled in the art to be suitable for the particular mode of administration. For example, solutions or suspensions used for parenteral, intradermal, subcutaneous, or topical application can include a sterile diluent (e.g., water for injection, saline solution, fixed oil, and the like); a naturally occurring vegetable oil (e.g., sesame oil, coconut oil, peanut oil, cottonseed oil, and the like); a synthetic fatty vehicle (e.g., ethyl oleate, polyethylene glycol, glycerine, propylene glycol, and the like, including other synthetic solvents); antimicrobial agents (e.g., benzyl alcohol, methyl parabens, and the like); antioxidants (e.g., ascorbic acid, sodium bisulfite, and the like); chelating agents (e.g., ethylenediaminetetraacetic acid (EDTA) and the like); buffers (e.g., acetates, citrates, phosphates, and the like); and/or agents for the adjustment of tonicity (e.g., sodium chloride, dextrose, and the like); or mixtures thereof. By further example, where administered intravenously, suitable carriers include physiological saline, phosphate buffered saline (PBS), and solutions containing thickening and solubilizing agents such as glucose, polyethylene glycol, polypropyleneglycol, and the like, and mixtures thereof.

By way of non-limiting example, tigecycline may be optionally combined with one or more pharmaceutically acceptable excipients, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solutions or suspensions containing from about 0.05 to 5% suspending agent in an isotonic medium. Such pharmaceutical preparations may contain, for example, from about 25 to about 90% of the active ingredient in combination with the carrier, more usually between about 5% and 60% by weight. Other formulations are discussed in U.S. Pat. Nos. 5,494,903 and 5,529,990, which are herein incorporated by reference.

The term “pharmaceutically acceptable salt” refers to acid addition salts or base addition salts of the compounds in the present disclosure. A pharmaceutically acceptable salt is any salt which retains the activity of the parent compound and does not impart any deleterious or undesirable effect on the subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Pharmaceutically acceptable salts may be derived from amino acids, including but not limited to cysteine. Other acceptable salts may be found, for example, in Stahl et al., Pharmaceutical Salts: Properties, Selection, and Use, Wiley-VCH; 1 st edition (Jun. 15, 2002).

Other than in the examples, and where otherwise indicated, all numbers used in the specification and claims are to be understood as modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The following examples are intended to illustrate the invention in a non-limiting manner.

### EXAMPLES

Nitration

Minocycline was prepared according to the method described in U.S. Pat. No. 3,226,436.

HPLC analyses were performed under the following conditions:

| | |
|---|---|
| Column: | Inertsil ODS3 5 μm, 25 × 0.46 cm |
| Mobile | 80% A + 20% B, where |
| Phase: | A = 90% (0.05 M KH₂PO₄ + 5 mL triethylamine/L |
| | phosphate + H₃PO₄ to pH 6)/10% Acetonitrile |
| | adjusted to pH 3.0 with H₃PO₄ |
| | B = Acetonitrile |
| Flow rate | 1.0 mL/min |
| Detection | 250 nm |

### Comparative Example 1

### Preparation of 9-nitrominocycline

This Example describes the nitration of minocycline where the product of the nitration was isolated.

13.44 grams of minocycline p-chlorobenzenesulfonate (i.e., [4S-(4alpha,12aalpha)]-4,7-bis(dimethylamino)-1,4,4a,5,5a,6,11,12a-octahydro-3,10,12,12a-tetrahydroxy-1,11-dioxo-2-naphthacenecarboxamide p-chlorobenzenesulfonate) was added slowly with stirring to 50 mL of concentrated sulfuric acid. The solution was cooled to 0-15° C. Nitric acid (90%, 0.6 mL) was added slowly and the solution was stirred at 0-15° C. for 1-2 h until the reaction was complete, as determined by HPLC. The solution containing the intermediate 9-nitrominocycline sulfate (i.e., [4S-(4alpha,12aalpha)-9-nitro]-4,7-bis(dimethylamino)-1,4,4a,5,5a,6,11,12a-octahydro-3,10,12,12a-tetrahydroxy-1,11-dioxo-2-naphthacenecarboxamide sulfate) was transferred with stirring to 300 g of ice and water over 20 min. The pH of the quench was adjusted to 5.0-5.5 with 28% aqueous ammonium hydroxide while maintaining the temperature between 0-8° C. The precipitate was filtered and washed with water (2×10 mL). The solid was dried under vacuum under a stream of nitrogen to give 9 g of crude 9-nitrominocycline sulfate.

Analysis (area %) by HPLC showed a purity of 90% with a C4-epimer content of 1.5%. MS(FAB): m/z 503 (M+H), 502 (M+). The product was isolated by precipitation at its isoelectric point from an aqueous solution. The crude sulfate molar yield was 45%.

Table 1 below lists data for other nitration processes:

TABLE 1

| | | minocycline | |
|---|---|---|---|
| Nitrominocycline | impurities | (μg/mg) | Molar yield (%) |
| A | 43.15 | 3.62 | 38 |
| B | 27.88 | 5.5 | 34 |

It can be seen that isolation of the 9-nitrominocycline resulted in a high amount of impurities.

### Comparative Example 2

### Preparation of 9-nitrominocycline

This Example describes the nitration of minocycline where the product of the nitration is isolated.

A 2-L multi-neck glass flask was equipped with a mechanical stirrer, thermocouple, liquid addition tube, nitrogen line, and gas outlet to a 30% (wt.) caustic scrubber. The flask was charged with sulfuric acid 66° Be (1,507 g, 819 mL, 15 moles). The solution was cooled to 0-2° C. Minocycline.HCl (92.7% potency, 311 g, 0.58 moles) was added to the sulfuric acid over 0.7 hours at 0-14° C. with stirring. After addition, the mixture was stirred at 0° C. for 0.5 hours to obtain a yellow solution. Nitric acid (95.9% nitrate content, 48 g, 32 mL, 0.73 moles, 1.25 mol equivalents) was added over 3 hours while keeping the mixture at 0-2° C. The mixture was stirred at 0° C. for 0.3 hours (dark red/black solution). Analysis (area %) by HPLC showed: 0% minocycline, 75.6% 9-nitrominocycline, 8.2% largest single impurity (LSI); relative retention time to minocycline (RRT)=2.08.

A 22-L multi-neck glass flask was equipped with a mechanical stirrer, thermocouple, and a condenser with nitrogen protection. The flask was charged with 6,704 g (8,540 mL) of isopropanol (IPA) and 1,026 g (1,500 mL) of heptanes. The solution was then cooled to 0-5° C. The 9-nitrominocycline reaction mixture was transferred to the 22-L flask over 2 hours at 0-39° C. to yield a yellow slurry. The slurry temperature was maintained at 34-39° C. for 2 hours then cooled to 20-34° C. and stirred at 20-34° C. for 14.6 hours.

A solution of isopropanol 3,028 g (3,857 mL) and heptanes 660 g (965 mL) was prepared and maintained at 20-25° C. (4:1, IPA:heptanes by volume). The slurry was filtered on a 30-cm diameter Buchner funnel using #1 Whatman filter paper under vacuum and nitrogen protection. The resulting wet cake was transferred to a 4-L glass Erlenmeyer flask, equipped with a mechanical stirrer and nitrogen protection. The cake was slurried by adding 1,608 mL of the prepared IPA/heptanes solution for 0.5 hours at 23-26° C.

The slurry was filtered again as described above. The wet cake was reslurried two more times as above (total of three reslurries). After the last filtration, the cake was maintained under vacuum under nitrogen protection for 0.2 hours. The product was dried at 40° C. under 23-11 mmHg of vacuum for 48 hours to a loss on drying (LOD, 80° C., 1 hour, >49 mmHg vacuum) value of 1.54. The weight of 9-nitrominocycline sulfate obtained was 380.10 g, HPLC strength=76.3% (as the disulfate salt), total impurities=34.6%, largest single impurity (LSI) 9.46% (RRT=0.94). Yield from minocycline.HCl=86%. Yield corrected for strength of product and starting material=71%.

It can be seen that isolating the 9-nitrominocycline compound resulted in a product having a large percentage of impurities.

### Example 1

Table 2 below outlines nitration experiments conducted using the procedure outlined in Comparative Example 2, where the following variables were modified: nitric acid addition time; reaction temperature; molar equivalents of nitric acid (relative to minocycline HCl); and agitation rate. In accordance with the methods disclosed herein, none of these reactions were quenched or worked up to isolate product. The sole analytical tool used was HPLC analysis.

TABLE 2

| HNO₃ | Reaction | | | | | | | | |
|---|---|---|---|---|---|---|---|---|---|
| Addition | Temp. | mol. Equiv. | Mino | 9-Nitro | Tot. Imp. | RRT 0.44 | RRT 0.51 | RRT 0.57 | RRT 1.23 |
| Time (hrs.) | (° C.)¹ | HNO₃ | (Area %) | (Area %) | (Area %) | (Area %) | (Area %) | (Area %) | (Area %) |
| 2 | 0 | 1.09 | 7.6 | 69.7 | 22.7 | 0.8 | 3.8 | 5.7 | 9.8 |
| 2.25 | 0 | 1.2 | 5.2 | 70.3 | 24.5 | 0.4 | 4.3 | 6.9 | 10.8 |
| 2.5 | 0 | 1.3 | 2.4 | 68.2 | 29.4 | 0.0 | 5.4 | 8.9 | 12.9 |
| 2.75 | 0 | 1.43 | 0.0 | 65.6 | 34.4 | 0.0 | 6.7 | 11.2 | 14.0 |
| 2 | 0 | 1.36 | 4.0 | 55.0 | 41.0 | 0.3 | 6.5 | 11.1 | 17.0 |
| 2.25 | 0 | 1.5 | 0.7 | 50.6 | 48.7 | 0.0 | 7.2 | 11.3 | 19.0 |
| 2.2 | 20 | 1.36 | 7.5 | 54.3 | 38.2 | 2.8 | 8.6 | 15.3 | 5.1 |
| 2.45 | 20 | 1.5 | 4.0 | 52.0 | 44.0 | 2.9 | 10.0 | 17.7 | 5.6 |
| 2.7 | 20 | 1.56 | 2.7 | 52.0 | 45.3 | 3.3 | 11.0 | 19.4 | 6.2 |
| 0.25 | 0 | 1.36 | 1.6 | 56.7 | 41.7 | 6.3 | 0.0 | 13.9 | 18.4 |
| 0.5 | 0 | 1.62 | 0.8 | 43.8 | 55.4 | 5.3 | 0.0 | 24.6 | 23.2 |
| 0.8 | 0 | 1.3 | 2.1 | 63.4 | 34.5 | 3.5 | 0.0 | 9.4 | 18.2 |
| 1 | 0 | 1.62 | 0.7 | 43.5 | 55.8 | 5.8 | 0.0 | 21.5 | 23.5 |
| 2.4 | 0 | 1.3 | 2.2 | 60.6 | 37.2 | 5.7 | 0.0 | 12.8 | 15.3 |
| 3 | 0 | 1.62 | 0.4 | 43.3 | 56.3 | 9.3 | 0.0 | 23.7 | 19.7 |
| 1.6 | 0 | 1.3 | 4.6 | 60.9 | 34.5 | 3.1 | 0 | 9.5 | 21 |
| 2 | 0 | 1.62 | 0 | 48.5 | 51.5 | 5.1 | 0 | 16.1 | 26.8 |
| 2.8 | 5 | 1.38 | 1.8 | 71.9 | 26.3 | 3.8 | 0 | 8 | 12.5 |
| 3.1 | 5 | 1.58 | 0 | 60 | 40 | 6.1 | 0 | 15.6 | 15.4 |
| 2.4 | 5 | 1.07 | 3.6 | 74.8 | 21.6 | 1.9 | 0 | 4.1 | 11.1 |
| 3 | 5 | 1.33 | 0 | 70 | 30 | 4.2 | 9.3 | 0 | 14.9 |

¹Only the bath temperature was monitored in these reactions due to vessel size.
² Reaction was at 50 wt % of original minocycline concentration.
³ Agitation was vigorous compared to all other experiments.
⁴ HNO₃ was added as 50 wt % in H₂SO₄.

It can be seen that despite the various conditions attempted, the amount of starting minocycline was present in an amount less than 10% and under certain conditions, was substantially removed.

### Example 2

Experiments were also performed that modified the nitration reaction, the reaction quench, and work up of the nitration reaction. The experiments were conducted using the procedure outlined in Comparative Example 2, modifying the following variables: nitric acid addition time; reaction temperature; molar equivalents of nitric acid (relative to minocycline HCl); temperature of the quench; composition of the quench solution; addition time of the reaction mixture to the quench solution; and wash method of the isolated cake. The data are shown in Table 3, below. The sole analytical tool used was HPLC analysis.

TABLE 3

| HNO₃ | Reaction | | Strength | | | | Quench | Quench | | |
|---|---|---|---|---|---|---|---|---|---|---|
| Addition | Temp. | mol. Equiv. | (di H₂SO₄ | Tot. Imp. | | Quench | Temp. | Add. | Wash | Yield |
| Time (hrs.) | (° C.)¹ | HNO₃ | salt, %) | (%) | LSI (%) | Comp.² | (° C.) | Time (hrs.) | Method³ | (corr., %)⁴ |
| 4.6 | 5 | 1.67 | 62.7 | 40.1 | 21.6 | IPA/hep | 0 | 0.1 | 1 | 60 |
| 4.6 | 5 | 1.67 | 61.0 | 39.7 | 18.8 | IPA/hep | 34 | 0.1 | 1 | 55 |
| 5.1 | 5 | 1.75 | 55.8 | 36.2 | 18.3 | IPA | 0 | 0.2 | 1 | 56 |
| 5.1 | 5 | 1.75 | 56.0 | 38.9 | 18.2 | IPA | 34 | 0.2 | 1 | 52 |
| 3 | 5 | 1.63 | 75.4 | 29.5 | 19.1 | IPA/hep | 0 | 1 | 1 | 70 |
| 3 | 5 | 1.63 | 74.8 | 27.8 | 18.9 | IPA/hep | 34 | 1 | 1 | 79 |
| 3 | 5 | 1.51 | 83.6 | 22.2 | 13.0 | IPA | 0 | 1 | 1 | 64 |
| 3 | 5 | 1.51 | 84.8 | 22.4 | 12.9 | IPA | 34 | 1 | 1 | 102 |
| 3.5 | −5 | 1.38 | 84.3 | 7.7 | 7.2 | IPA/hep | 0⁵ | 2 | 1 | 96 |
| 3.5 | −5 | 1.38 | 101.8 | 11.4 | 8.3 | IPA/hep | 0⁵ | 2 | 2 | 104 |

¹Only the bath temperature was monitored in these reactions due to vessel size.
²When IPA was used as the quench, heptanes were then added to obtain the composition of the original quench mixture.
³Wash method 1: wet cake was washed on the filter with 4:1 IPA:hep. (vol.). Wash method 2: wet cake was slurried three times with 4:1 IPA:hep. (vol.). Wash method #2 used 20% more wash solution than method #1.
⁴Yield is corrected for strength of the product and starting material.
⁵The quench was started at 0° C. then immediately heated to 34° C. and held at 34° C. for the remainder of the quench.

It can be seen from the data of Table 3 that the yield was at least 50%.

### Example 3

This Example shows the results of varying the amount of nitric acid (in equivalents) needed for the nitration step. The nitric acid was titrated at 89.5% and amount used corrected accordingly.

Three trials were performed. Trial 1 used 1.25 equivalent nitric acid, Trial 2 used 1.09 equivalent, and Trial 3 used 1.00 equivalent nitric acid.

The HPLC completion test of Trial 1 showed no signal of minocycline while completion test for Trial 2 showed 2.5% unreacted starting material. Both reactions were hydrogenated and then converted to the aminominocycline hydrochloride salt using the SLP procedure.

Hydrogenated product 1 (from Trial 1) showed a minocycline content of 0.37%; Strength=83.0%, total imp.=3.20%; single imp.=0.52%; epimer content=1.1%

Hydrogenated product 2 (from Trial 2) showed aminocycline content of 1.6%; Strength=84.2%; total imp.=4.00%; single imp.=0.35%; epimer content=1.0%.

Trial 3: Strength=83.0%; total imp.=5.0%; single imp.=2.7%; epimer content=1.1%.

Reduction

HPLC analyses were performed under the following conditions:

| | |
|---|---|
| Column: | Inertsil ODS3 5 μm, 25 × 0.46 cm |
| Mobile | 80% A + 20% B, where |
| Phase: | A = 90% (0.05 M KH₂PO₄ + 5 mL triethylamine/L |
| | phosphate + H₃PO₄ to pH 6)/10% Acetonitrile |
| | adjusted to pH 6.0 with H₃PO₄ |
| | B = Acetonitrile |
| Flow rate | 1.0 mL/min |
| Detection | 250 nm |

### Example 1

This Example describes a hydrogenation reaction where the 9-nitrominocycline intermediate was not isolated.

10.1 grams of minocycline p-chlorobenzenesulfonate was added slowly with stirring to 27 mL of concentrated sulfuric acid. The solution is cooled to 0-2° C. Nitric acid (0.6 mL, 90%) was added slowly and the solution was stirred at 0-2° C. for 1-2 h until the reaction was complete as determined by HPLC. After the nitration was complete, the solution containing the intermediate 9-nitrominocycline sulfate was transferred with stirring to 150 mL of isopropanol and 1200 mL of methanol while keeping the temperature below 10-15° C. The solution was hydrogenated at 26-28° C. at 40 psi for 3 h in the presence of 10% Pd on carbon catalyst, which was 50% wet. After hydrogenation was complete, the catalyst was filtered off and the solution was slowly poured into 250 mL isopropanol with stirring at 0-5° C. The solid (3.4 g) was filtered off. Crude purity by HPLC (area %) was 90%. C₄-epimer was present in an amount of 0.9%). MS(FAB): m/z 473 (M+H), 472 (M+).

### Example 2

This Example describes a hydrogenation reaction where the 9-nitrominocycline intermediate was not isolated.

84.3 grams of minocycline p-chlorobenzenesulfonate was added slowly with stirring to 368 g of concentrated sulfuric acid. The solution was cooled to 10-15° C. Nitric acid (6.0 mL, fuming) was added slowly. The solution was stirred at 10-15° C. for 1 to 2 h until the reaction is complete, as determined by HPLC. After the nitration was complete, the solution containing the intermediate 9-nitrominocycline sulfate was transferred with stirring to 0.3 Kg of methanol while keeping the temperature below 10-15° C. The solution was hydrogenated at 26-28° C. at 50 psi for 2-3 h in the presence of 10% Pd on carbon catalyst, which was 50% wet. After hydrogenation was complete, the catalyst was filtered off and the solution was slowly poured into 0.6 kg of isopropanol and 0.3 Kg of n-heptane with stirring at 0-5° C. The solid was filtered off.

The wet solid was dissolved in 100 g of water at 0-5° C. The mixture was stirred and the organic phase was separated and discarded. To the aqueous phase was added 14.4 g of concentrated HCl. The pH of the solution was adjusted to 4.0±0.2 with ammonium hydroxide. 100 mg of sodium sulfite was added and the solution was seeded with 100 mg of 9-aminominocycline. The mixture was stirred for 4 h at 0-5° C. and the product was filtered and dried to give 28.5 g of solids. Purity by HPLC (area %) was 96.5%, with 0.9% C4-epimer. MS(FAB): m/z 473 (M+H), 472 (M+). Yield: 54.2%.

### Comparative Example 1

This Example describes a hydrogenation reaction where the 9-nitrominocycline intermediate was isolated.

52.0 kg of minocycline.HCl (92.4% potency) was charged to 4.8 parts (251 kg) sulfuric acid 66° Be at 0 to 15° C. in a 300 gallon vessel and stirred to effect removal of HCl. 7.48 kg of nitric acid, fuming 100% (95.9% nitrate content, 1.26 equivalents) was charged over 3 hours and 20 minutes.

HPLC analysis indicated >1% minocycline remained. Accordingly, 0.31 kg of nitric acid, fuming 100% (95.5% nitrate content, 0.05 equivalents) was added. HPLC analysis still indicated >1% minocycline remained. Another 0.74 kg of nitric acid, fuming 100% (95.5% nitrate content, 0.12 equivalents) was added. As HPLC testing once again indicated >1% minocycline remained, another 1.11 kg of nitric acid, fuming 100% (95.5% nitrate content, 0.19 equivalents) was added, after which <1% minocycline remained.

The nitration reaction mixture was transferred to a solution of 21.5 parts IPA/3.3 parts heptane (1120 kg IPA/171 kg heptane) at 0 to 36° C. The slurry was filtered (lengthy filtration time), washed with IPA/heptane 4:1 and dried at NMT 40° C. to an LOD of NMT 6%, yielding 70.9 kg of sulfate salt (97% crude yield) for use in reduction reaction.

### Example 3

This Example describes a hydrogenation reaction where the 9-nitrominocycline intermediate was not isolated.

25.0 kg of minocycline.HCl (94.4% potency) was charged to 7.3 parts (183 kg) sulfuric acid 66° Be at 5 to 15° C. in a 100 gallon vessel and stirred to effect removal of HCl. 2.5015 kg of nitric acid, 85% (86.6% nitrate content, 1.25 equivalents) was added to the vessel over 78 minutes at 9 to 15° C.

HPLC analysis indicated >1% minocycline remained. Another 0.261 kg nitric acid, 85% (86.6% nitrate content, 0.13 equivalents) was added. As HPLC once again indicated >1% minocycline remained, another 0.261 kg nitric acid, 85% (86.6% nitrate content, 0.13 equivalents) was added. As HPLC still indicated >1% minocycline remained, another 0.174 kg nitric acid, 85% (86.6% nitrate content, 0.09 equivalents) was added, after which it appeared the reaction reached a plateau at 1.7% minocycline starting material.

The nitration reaction mixture was transferred to 4.2 parts (106 kg) methanol at −20 to 10° C. The quenched batch was adjusted to 4 to 10° C. and used as-is in the reduction reaction.

### Comparative Example 2

This Example describes a hydrogenation reaction where the 9-nitrominocycline intermediate was isolated.

104 kg minocycline.HCl (90.3% potency) charged to 4.8 parts (502 kg)sulfuric acid 66° Be at 0-10° C. in a 300 gallon vessel and stirred to effect removal of HCl. 15.2 kg fuming nitric acid (100.4%, 1.25 equivalents) charged over 3 hours at 0-6° C., 100 rpm. As HPLC testing indicated >1% minocycline remained, another 0.69 kg fuming nitric acid (100.4%, 0.06 equivalents), was added, after which minocycline <1%. The nitration mixture was transferred to a solution of 21.5 parts IPA/3.3 part heptane at 0-36° C.

The slurry was filtered (lengthy filtration time), washed with IPA/heptane 4:1 and dried at NMT 40° C. to an LOD of NMT 6%, yielding 140 kg of sulfate salt (95% crude yield) for use in reduction reaction.

### Example 4

This Example describes a hydrogenation reaction where the 9-nitrominocycline intermediate was not isolated.

104 kg minocycline HCl (90% potency) charged to 7.3 parts (762 kg) sulfuric acid 66° Be at 5-15° C. and stirred to effect removal of HCl. 14.9 kg fuming nitric acid (100%, 1.25 equivalents) was charged over 1 hour at 5-15° C., 120 rpm. As HPLC analysis indicated that >1% minocycline remained, another 0.69 kg fuming nitric acid (100%, 0.06 equivalents), was added after which minocycline <1%.

The nitration mixture was transferred to 4.2 parts (440 kg) methanol at −10 to −20° C. The quenched batch was adjusted to 4-10° C. and used as is in the reduction reaction.

### Comparative Example 3

This Example describes a hydrogenation reaction where the 9-nitrominocycline intermediate was isolated. Proportions of solvents/reagents are relative to the initial charge of minocycline prior to nitration reaction.

The 9-nitrominocycline sulfate reaction mixture of Comparative Example 4 was quenched into 2240 kg (21.5 parts) isopropanol and 342 kg (3.3 parts) heptane, over 1 hour, while maintaining the batch temperature at 0 to 36° C. The resulting slurry was stirred at 30 to 36° C. for 2 hours, then cooled and stirred at 19 to 25° C. for 1 hour. One half of the slurry was filtered, washed with 3×205 kg IPA/heptane (4:1) v/v and dried at NMT 40° C. to an LOD of NMT 6%. Filtration and drying took 16 days (for 7 of these days the wet cake was idle under nitrogen during a scheduled plant shutdown) and yielded 58 kg of sulfate salt. The second half of the slurry was drummed and refrigerated pending filter availability. It was refrigerated for 12 days, then charged back to the vessel and stirred at 0 to 6° C. for 2 days, then adjusted to 19 to 25° C., filtered, washed with 3×205 kg IPA/heptane (4:1) v/v and dried at NMT 40° C. to an LOD of NMT 6%. Filtration and drying took 6 days and yielded 82 kg of sulfate salt.

Both sub-lots of 9-nitrominocycline sulfate were dissolved in 672 kg (6.5 parts) methanol and 8.4 kg (0.08 parts) water for injection, USP at 19 to 25° C. and reduced to 9-aminominocycline sulfate using 70 psig hydrogen gas and 2.74 kg (0.026 parts) Palladium on Carbon, wet 10% (w/w). The hydrogenation reaction took 10.5 hours and resulted in no detectable starting material.

The 9-aminominocycline sulfate reaction mixture was filtered to remove catalyst and quenched into a solution of 1660 kg (16 parts) IPA/710 (6.8 parts) heptane at 0 to 27° C., over 1 hour. The resulting mixture was adjusted to 19 to 25° C. and stirred for 1 hour.

The 9-aminominocycline sulfate slurry was filtered on a Nutsche filter, washed with 2×162 kg (1.5 parts each) IPA/heptane (4:1) v/v and dried at 40° C. to an LOD of less than 4%. The filtration, washing and drying took 10 days and gave 94.0 kg of 9-aminominocycline sulfate. After filtration, solids were observed in the mother liquors. These were filtered, washed with 113 kg IPA/heptane (4:1) v/v and dried at 40° C. to an LOD of less than 4%. 24.1 kg were recovered and retained as a separate lot. Total crude yield of 9-aminominocycline sulfate from minocycline was 84%.

The 94.0 kg ‘1^{st} crop’ of dried 9-aminominocycline sulfate and 0.084 kg (0.0008 parts) sodium sulfite were dissolved in 538 kg (5.17 parts) water for injection, USP and cooled to 0 to 6° C., 0 kg hydrochloric acid, 20° Be was required to bring the 9-aminominocycline sulfate solution pH to 1.1+/−0.1 because the initial pH was 1.16. 48.3 kg (0.46 parts) of hydrochloric acid, reagent was added to the 9-aminominocycline solution, forming 9-aminominocycline HCl. 56 kg (0.54 parts) of ammonium hydroxide, 28% and 4.0 kg (0.039 parts) hydrochloric acid, reagent were added to the solution to obtain a batch pH of 4.0+/−0.2.

The batch was then stirred for 90 minutes at 0 to 6° C. while ensuring the pH stayed at 4.0+/−0.2. The final pH reading was 4.05 pH units. The batch was filtered on a Nutsche filter, washed with 2×33 kg (0.3 parts each) water for injection (pH'ed to 4.0) pre-cooled to 2 to 8° C., followed by 2×26.1 kg (0.25 parts acetone (pre-cooled to 2 to 8° C.) and dried at NMT 40° C. to a moisture content of NMT 7.0%. 43.2 kg of 9-Aminominocycline HCl was isolated, a 40% yield from minocycline HCl.

Processing of the 24.1 kg ‘2^{nd} crop’ of dried 9-aminominocycline sulfate through the salt change proceeded similarly to the process as described in the previous four paragraphs using proportional quantities of reagents. An additional 9.9 kg of 9-Aminominocycline HCl were recovered representing an incremental additional yield of 9.2%. The total batch yield including both crops was 53.1%.

### Example 5

This Example describes a hydrogenation reaction where the 9-nitrominocycline intermediate was not isolated. Proportions of solvents/reagents are relative to the initial charge of minocycline prior to nitration reaction.

The 9-nitrominocycline sulfate reaction mixture from Example 7 was transferred into 440 kg (4.2 parts) of methanol, over 90 minutes, while maintaining the batch temperature at −20 to −10° C. and the agitation rate at 130 RPM.

The quenched batch was adjusted to 4 to 10° C. and reduced to 9-aminominocycline sulfate using 50 psig hydrogen gas and 52 kg (0.5 parts) Palladium on Carbon, wet 10% (w/w). The hydrogenation reaction took 5 hours and resulted in no detectable starting material. The 9-aminominocycline sulfate reaction mixture was filtered to remove catalyst and quenched into a solution of 1241 kg (12 parts) IPA/537 kg (5.2 parts) heptane at 17 to 23° C., over 30 minutes. The resulting mixture was then cooled to −18 to −12° C. and stirred for 1 hour.

The resulting 9-aminominocycline sulfate slurry was filtered on a Nutsche filter in two portions and washed with a total of 3.6 parts IPA/heptane (2:1) v/v pre-cooled to 0 to 6° C. and 506 kg (4.9 parts) cold heptane. The filtration and washing took 99 hours for both portions (filtered in two portions due to size limitation of the filter). The 9-aminominocycline sulfate wet cakes were dissolved in 150 kg (1.4 parts) water for injection, USP at 0 to 6° C. and the upper organic layer separated off as waste.

25.7 kg (0.3 parts) hydrochloric acid, 20° Be was added to the 9-aminominocycline sulfate solution at 0 to 6° C. for conversion to 9-aminominocycline HCl. Ammonium hydroxide, 28% was added to the reaction mix to obtain a batch pH of 4.0+/−0.2; this took 49.5 kg (0.48 parts). 0.15 kg Sodium sulfite (0.0014 parts) was added to the reaction mixture.

The batch was seeded with 5 g of 9-aminominocycline HCl and stirred for 3 hours while maintaining the pH at 4.0+/−0.2 using ammonium hydroxide, 28% (took 0.05 parts). The batch was filtered on a Nutsche filter, washed with 1 part water for injection (ph'ed to 4.0) pre-cooled to 2 to 8° C., followed by 0.2 parts isopropanol (pre-cooled to 2 to 8° C.) and dried at NMT 50° C. to an LOD of NMT 10.0% and a moisture content of NMT 8.0%.

63.1 kg of 9-Aminominocycline HCl was isolated, a 59% yield from minocycline HCl.

Table 4 below lists the Comparative Data.

TABLE 4

| | | | Strength | | Single | | | |
|---|---|---|---|---|---|---|---|---|
| | Scale (kg | | corrected | Total | Largest | | Cycle | |
| Batch | minocycline HCl) | Strength | yield | Impurities | Impurity | Epimer | Time¹ | |
| (Example 3) | 30 kg | 84.1% | 40.3% | 4.49% | 2.76% | 2.76% | 8 | days |
| (Comp. Example 1 ) | 52 kg | 90.4% | 37.0% | 6.45% | 0.84% | 1.75% | 24 | days |
| | 52 kg | 87.9% | 27.2% | 9.72% | 3.73% | 3.88% | 25 | days |
| (Comp. Example 2 | 104 kg | 86.4% | 48%² | 10.79% | 0.63% | 3.18% | 33 | days³ |
| or 3) | | | | | | | | |
| | | 87.8% | | 9.31% | 0.57% | 2.46% | | |
| (Example 4 or 5) | 104 kg | 87.7% | 57% | 3.5% | 1.2% | 0.72% | 14 | days |

¹cycle time is from minocycline.HCl to 9-aminominocycline HCl.
²combined yield of 1^{st} and 2^{nd} crops
³Does not include 7 day plant shutdown that occurred during process, does include time to process 2nd crop.

Table 4 indicates that hydrogenation of a reaction mixture without isolation results in a product with a lower amount of impurities and C₄-epimer.

Acylation

HPLC analyses were performed under the following conditions:

| | |
|---|---|
| Column: | Luna C8 5 μm, 15 × 0.46 cm |
| Mobile | 80% (0.05 M KH₂PO₄ + 10 mL triethylamine/L |
| Phase: | phosphate + H₃PO₄ to pH 6.2)/20% |
| | Acetonitrile + 0.5 g NaEDTA |
| Flow rate | 1.0 mL/min |
| Detection | 250 nm |

### Example 1

### N-t-Butylglycine Hydrochloride

To a mixture of t-butyl amine (1.57 L) and toluene (1.35 L) at 45-50° C. is added t-butyl bromoacetate (420 mL). The mixture is stirred for 1 h at 50-60° C., the temperature is increased to 75° C. over 1 h. After 2 h at 75° C., the mixture is cooled to −12±3° C. and let stand for 1 h. The solid is collected by filtration, and the filtrate is concentrated by distillation (30-40° C., 25-35 mm Hg) to a volume of 825 mL. The resulting concentrate is cooled to 20-25° C. and 6N HCl (1.45 kg) is added. After 3 h, the phases are separated and the aqueous phase is concentrated by distillation (30-40° C., 25-35 mm Hg) to a volume of 590 mL. Isopropanol (2.4 L) is added and the mixture is concentrated by distillation (15-20° C., 10-20 mm Hg) to a volume of 990 mL. The resulting slurry is cooled to −12±3° C. over 30 min. and let stand for 1 h. The solid is collected by filtration, washed with i-PrOH, and dried (45±3° C., 10 mm Hg) for 24 h to afford (407.9 g, 86%) of the desired product.

### Example 2

### N-t-Butylglycine Acid Chloride Hydrochloride

To a mixture of milled N-t-butylglycine hydrochloride (250.0 g), toluene (1.14 L), and DMF (7.1 g) is added thionyl chloride (143 mL) over 20 min. The mixture is brought to 80-85° C. and heated with stirring for 3 h. After cooling to 20° C., the solid is collected by filtration under N₂, washed with toluene, and dried (40° C., 10 mm Hg) for 16 h to afford the desired product (260.4 g, 93.8%). Purity by HPLC area %: 98.12%

### Example 3

### Tigecycline

To a mixture of 9-aminominocyline.HCl (140.0 g) and cold (0-4° C.) water (840 mL) is added N-t-butylglycine acid chloride hydrochloride (154.0 g) over 15 min with stirring. The mixture is stirred at 0-4° C. for 1-3 h. Ammonium hydroxide (126 g, 30%) is added to bring the pH to 7.2 while maintaining the temperature at 0-10° C. Methanol (930 mL) and CH₂Cl₂ (840 mL) are added and the mixture is stirred at 20-25° C. for 1 h, while maintaining the pH at 7.2 by addition of ammonium hydroxide (13.5 g, 30%). The phases are separated, and the solids are combined with the organic layer. The aqueous layer is extracted with CH₂Cl₂ (1×840 mL, 3×420 mL) and the pH of the mixture is adjusted to 7.2 during each extraction. To the combined organic layers is added methanol (200 mL) to afford a solution. The solution is washed with water (2×140 mL), then dried over sodium sulfate (140 g) with stirring for 30 min. The mixture is filtered and the filtrate is concentrated by distillation (20° C., 15-25 mm Hg) to a volume of 425 mL. To this mixture is added CH₂Cl₂ (1.4 L) and the distillation is repeated two times. The resulting suspension is cooled to 0-2° C. and stirred for 1 h. The solid is collected by filtration, washed with 0-5° C. CH₂Cl₂ (2×150 mL), and dried (65-70° C., 10 mm Hg) for 24 h to afford of the desired product (120.0 g, 75%). Purity by HPLC area %: 98.9% and C-4 epimer 0.12%.

### Example 3A

### Tigecycline

To a mixture of 9-aminominocyline.HCl (100.0 g) and cold (0-4° C.) water (600 mL) was added N-t-butylglycine acid chloride hydrochloride (110.0 g) over 50 min with stirring. The mixture was stirred well at 0-4° C. for 1.5 h. Ammonium hydroxide (112 g, 28%) was added to bring the pH to 7.2 while maintaining the temperature at 0-5° C. Methylene chloride (600 mL), then methanol (440 mL) were added and the mixture was stirred at 0-5° C. for 30 min, while maintaining the pH at 7.2 by addition of ammonium hydroxide (10.0 g, 28%). The mixture was warmed to 20-25° C. over 15 min. Methanol (244 mL) was added and the phases were separated. The aqueous layer was extracted with CH₂Cl₂ (1×600 mL, 3×300 mL) and the pH of the mixture was adjusted to 7.2 during each extraction. To the combined organic layers was added methanol (144 mL) to afford a solution. The solution was washed with water (2×100 mL), then dried over sodium sulfate (100 g) with stirring for 30 min. The mixture was filtered and the filtrate was concentrated by distillation (20° C., 80-120 mm Hg) to a volume of 400 mL. To this mixture was added CH₂Cl₂ (1.0 L) and the distillation was repeated two times. The resulting suspension was cooled to 0-2° C. and stirred for 1 h. The solid was collected by filtration, washed with 0-5° C. CH₂Cl₂ (2×110 mL), and dried (65-70° C., 20 mm Hg for 18 h, then 3-5 mm Hg for 16 h) to afford the desired product (82.4 g, 71.7%). Purity by HPLC area %: 98.5% and C-4 epimer 0.28%.

### Example 4

### N-t-Butylglycine Acid Chloride Hydrochloride

t-Butylamine (88 g) was dissolved in 300 mL of toluene. The mixture was heated to 45-50° C. and 117.5 g of t-butylbromoacetate was added over 1 h while maintaining the temperature at 50-60° C. The mixture was heated to 75° C. for 2 hours. The reaction mixture was then cooled to 12-15° C. and stirred for 1 hour. The solids were filtered off and washed with cold toluene. The solid which was t-butylamine hydrobromide was discarded. The filtrate was cooled to 10-12° C. and HCl gas was bubbled in for 0.5 h. The mixture was stirred for 3 h at 10-12° C., then the product was collected by filtration and washed with cold toluene. The product was dried under vacuum at 40-50° C. to give 107 g of N-t-butylglycine hydrochloride. MS: m/z 187 (M+)

N-t-butylglycine hydrochloride (7 g) from the material prepared as described above was added to 35 mL of toluene. Thionyl chloride (11.6 mL) was added and the slurry was heated at 75-80° C. for 1 h. The suspension is cooled to 20° C. and the solid is collected by filtration and washed with 2×15 mL of toluene. The resulting solid is dried under vacuum at 40° C. to afford 4.4 g (65% yield) of product, which is protected from moisture and used immediately in the next step.

### Example 5

### Tigecycline

9-Aminominocycline (10.00 g) was added portion-wise to 60 mL of water at 0-5° C. t-Butylglycine acid chloride hydrochloride (10.98 g) was added portion-wise keeping the temperature at 0-5° C. After stirring for 40-60 min., 30% ammonium hydroxide was added dropwise to the reaction mixture while keeping the temperature at 0-5° C. to adjust pH to 7.2. To the solution was added 83 mL methanol followed by 60 mL methylene chloride. After stirring for 15 min., the phases were separated. The aqueous phase was extracted with 4×40 mL methylene chloride adjusting pH to 7.2 before each extraction. To the combined organics was added 10 mL methanol, and the solution was dried over sodium sulfate. After filtering, the solution was concentrated to give a suspension (net weight 51 g). The suspension was stirred at 5-10° C. for 1 h then filtered. The solid was washed with 2×10 mL cold methylene chloride, then dried to give 8.80 g of product (76.8% yield). Purity by HPLC area %: 98.4% and C-4 epimer 0.1%. MS(FAB): m/z 586 (M+H); 585 (M⁺).

### Example 6

### N-t-Butylglycine Acid Chloride Hydrochloride

t-Butylamine (1.5 kg) was dissolved in 1.35 L of toluene. The mixture was heated to 45-50° C., and 548 g of t-butylbromoacetate is added over 1 h while maintaining the temperature at 50-60° C. The mixture was heated at 75° C. for 3 h. The reaction mixture was then cooled to 12-15° C. and stirred for 1 h. The solids were filtered off and washed with cold toluene. The solid which was t-butylamine hydrobromide was discarded. The filtrate was concentrated to ˜800 mL by distilling off the solvent. The concentrate was cooled to 25° C. and 900 mL of 6N HCl was added to the mixture. After stirring for 3 h at 20 to 25° C., the phases were separated. The organic phase was discarded and the aqueous phase was concentrated to a volume of 600 mL. Isopropanol (2.4 L) was added to the concentrate. The slurry was cooled to −12 to −9° C. and held for 0.5 h. The product was collected by filtration, washed with cold isopropanol, then dried under vacuum at 40-50° C. to give 408 g of solid. Purity by NMR >95%. MS: m/z 187 (M+).

N-t-butylglycine hydrochloride (250 g) from the material prepared as described above was added to 1.3 L of toluene and 7.5 mL of DMF. Thionyl chloride (143 mL) was added and the slurry is heated at 80-85° C. for 3-4 h. The suspension was cooled to 20° C. and the solid was collected by filtration and washed with 2×250 mL of toluene. The solid was dried in vacuum at 40° C. to afford 260 g (82% yield) of product. Purity by HPLC area %: 98.2%

### Example 7

### Tigecycline

9-Aminominocyline.HCl (140.0 g) was added portion-wise to 840 mL of water at 0-4° C. t-Butylglycine acid chloride hydrochloride (154 g) was added over 15 min with good stirring while maintaining the temperature at 0-4° C. The solution was stirred for 1-3 h. The pH of the mixture was adjusted to 7.2±0.2 with 30% ammonium hydroxide while maintaining the temperature at 0-10° C. Methanol (930 mL) and 840 mL of methylene chloride were added to the solution, which was stirred for 1 h at 20-25° C. The phases were separated. The aqueous phase was extracted with 3×600 mL of methylene chloride, and the organic phases were combined, dried and concentrated to a volume of approximately 500 mL. The resulting suspension was cooled to 0-2° C. for 1 h. The solid was filtered and dried to give 120 g of product (75% yield). Purity by HPLC area %: 98%, C-4 epimer 0.1%. MS(FAB): m/z 586 (M+H); 585 (M⁺).

### Example 8

### Pyrrolidinylacetic Acid Hydrochloride

Pyrrolidine (14.2 g) was dissolved in 40 mL of methyl t-butyl ether. The solution was cooled to 0 to −5° C. Benzyl bromoacetate (22.9 g) was added dropwise with stirring. The thick white slurry was stirred for 0.5 h at 0-5° C. The solid was filtered off and washed with methyl t-butyl ether. The filtrate was concentrated to give 21.3 g of pyrrolidinylbenzyl acetate. The benzyl ester (21.0 g) was dissolved in 200 mL of methanol and 4.0 g of 10% Pd/C catalyst (50% wet) was added. The solution was hydrogenated at 40 psi for 6 h. The catalyst was filtered off and washed with methanol. The filtrate was concentrated to give 11.8 g of pyrrolidinyl acetic acid as a colorless oil. 15.8 g pyrrolidinyl acetic acid was slurried in 15 mL of methyl-t-butyl ether. Acetonitrile (15 mL) was added and the suspension is cooled to 0-5° C. Ethereal HCl (120 mL, 1.0 M) was added with stirring. The resulting white precipitate was filtered, washed with methyl t-butyl ether, and dried to give 15 g of pyrrolidinyl acetic acid hydrochloride. Purity by GC/MS area %: 98%. MS: m/z 129 (M+).

### Example 9

### [4S-(4α,12aα)]-4,7-Bis(dimethylamino)-9-[(pyrrolidinyl)acetyl]amino]-1,4,4a,5,5a,6,11,12a-octahydro-3,10,12,12a-tetrahydroxy-1,11-dioxo-2-naphthacene-carboxamide

Pyrrolidinylacetic acid (7.7 g) was suspended in 7 mL of acetonitrile. After cooling to 0-5° C., 5.3 mL of thionyl chloride was added slowly with stirring. The suspension was heated to 55° C. The dark solution was kept at 55° C. for 0.5 h and then cooled to room temperature to afford pyrrolidinylacetyl chloride hydrochloride. 9-Aminominocycline hydrochloride (5.0 g), prepared as described in Example 4 above, was suspended in 5.0 mL of water. The suspension was cooled to −15° C. To this suspension was added dropwise the solution of pyrrolidinylacetyl chloride hydrochloride prepared as described above, keeping the temperature below 22° C. The dark reaction mixture was stirred at 22-25° C. for 3 h. Water (2 mL) was added to the mixture, and the pH was adjusted to 6.5±0.2 with 30% ammonium hydroxide. The solution was extracted with 6×15 mL of CH₂Cl₂. The organic extracts were pooled and concentrated at 40° C. Anhydrous ethanol (10 mL) was added to the concentrate, and the slurry was stirred at 5-7° C. for 1 h. The solid was filtered and dried in vacuum at 40° C. to afford 3.5 g of product. Purity by HPLC area %: 98.7%, C-4 epimer 0.4%. MS(FAB): m/z 586 (M+H); 585 (M⁺).

### Example 10

### Tigecycline

9-Aminominocycline (4.0 g) was added portion-wise to 10 mL of acetonitrile and 5 mL of DMPU at 10-15° C. t-Butylglycine acid chloride hydrochloride (4.4 g) was added portion-wise keeping temperature at 10-15° C. After stirring for 2 h, 10 mL MeOH and 17 mL of water was added slowly to the reaction mixture maintaining temperature between 10-17° C. Ammonium hydroxide (30%) was added dropwise to the reaction mixture, keeping the temperature at 5-8° C., to adjust pH to 7.2. To the solution was added 15 mL methylene chloride. After stirring for 15 min., the phases were separated. The aqueous phase was extracted with 2×20 mL of methylene chloride, adjusting pH to 7.2 before each extraction. To combined organics was added 700 mg of Norit CA-1 (charcoal) and 10 g sodium sulfate, then the mixture was filtered. The cake was washed with 2×20 mL of methylene chloride. The solution was concentrated and the resulting suspension was stirred at 5-8° C. for 16 h. After filtering, the solid was washed with 2×10 mL cold methylene chloride, then dried to give 2.3 g of product (50% yield). Purity by HPLC area %: 95.2%, C-4 epimer: 0.5%. MS(FAB): m/z 586 (M+H); 585 (M⁺).

### Examples 11-19

### Tigecycline

Examples 11-19 followed the procedure of Example 10 with the solvent modification as indicated below.

| Example | Solvent | Yield | Results¹ |
|---|---|---|---|
| 11 | DMPU | 50% | Purity: 95.2%, C-4 |
| | | | epimer: 0.5%, sm: 3.35% |
| 12 | DMPU- | 48% | Purity: 98.1%, C-4 |
| | H₂O (1:1) | | epimer: 0.5%, sm: 0.7% |
| 13 | DMPU- | 60-72% | required extensive workup |
| | MeCN | yield, 6 g | |
| | | scale | |
| 14 | THF | — | Reaction “does not |
| | | | go to completion.” |
| 15 | MeCN | — | “incomplete reaction” |
| 16 | CH₂Cl₂ | — | “incomplete reaction” |
| 17 | THF:H₂O | — | Reaction was not successful |
| | (6:1) | | |
| 18 | NMP | — | Reaction “works well |
| | | | on small scale,” “led to a |
| | | | complete reaction” |
| 19 | DMF² | 58% | Unknown impurity: 1.5% |

¹Purity assessed by HPLC area. sm = starting material 9-Aminominocyline.
²Reaction mixture was quenched with isopropanol-ethyl acetate, then partitioned between water and CH₂Cl₂. The organic phase was concentrated, then diluted with toluene prior to isolation of the product.

### Example 20

### N-t-Butylglycine Acid Chloride Hydrochloride

To a 5-L multi-neck flask with a mechanical stirrer, thermocouple, condenser with a nitrogen line to a 30% (wt.) caustic scrubber, and a 250-mL pressure equalizing addition funnel was added the milled N-t-butylglycine hydrochloride (436 g, 2.60 moles, d(0.5)=103 μm), toluene (1,958 g, 2,263 mL), and N,N-dimethylformamide (13.6 g, 14.4 mL, 0.19 moles). Thionyl chloride (405 g, 248 mL, 3.40 moles) was added to the off-white slurry, using the 250-mL addition funnel over 33 min at 20-23° C. The slurry was slowly heated to 80° C. over 1 hour, then stirred at 80° C. for 3 hours. After 3 hours the reaction was complete by thin layer chromatography (<2% starting material). The yellow-orange suspension was cooled to 20° C. over 32 min., then stirred at 15-20° C. for 32 min. The solid was collected by vacuum filtration on a 15-cm Buchner funnel using #42 Whatman paper. The cake was washed with three portions of toluene (272 g, 314 mL each wash) at 20-25° C. The wet caked was dried with suction for 20 minutes under nitrogen protection. The product was then dried in an oven with a vacuum of 23 mm Hg and 38° C. for 21.2 hours to yield a loss on drying of 1.23%. Weight of t-butylaminoacetyl chloride HCl obtained=462 g, GC strength=91.0%, IR identification=positive. Yield from t-butylaminoacetic acid HCl=96%. Yield corrected for strength of product and starting material=87%.

### Example 21

### N-t-Butylglycine Acid Chloride Hydrochloride

To a 5-L multi-neck flask with a mechanical stirrer, thermocouple, condenser with a nitrogen line to a 25% (wt.) caustic scrubber, and a 250-mL pressure equalizing addition funnel was added the milled N-t-butylglycine hydrochloride (450 g, 2.68 moles, d(0.5)=664 μm), toluene (2,863 g, 3,310 mL), and N,N-dimethylformamide (15 g, 15 mL, 0.21 moles). Thionyl chloride (422 g, 259 mL, 3.54 moles) was added to the white slurry, using the 250-mL addition funnel over 19 min at 19-22° C. The slurry was slowly heated to 79° C. over 7.1 hours, then stirred at 79-82° C. for 44 hours. The reaction was checked at 3 hours and found to be incomplete by thin layer chromatography (TLC). An additional 26 mL (42 g, 0.35 moles) of thionyl chloride was added. After a total of 27 hours, the reaction was still incomplete by TLC and an additional 26 mL (42 g, 0.35 moles) of thionyl chloride was added. After a total of 44 hours, at 79-82° C., the reaction was complete by TLC (<4% starting t-butylaminoacetic acid HCl). The dark brown suspension was cooled to 25° C. over 17 min., then stirred at 21-25° C. for 37 min. The solid was collected by vacuum filtration on a 2-L coarse glass sintered funnel. The cake was washed with six portions of toluene (282 g, 325 mL each wash) at 20-25° C. The wet caked was dried with suction for 16 minutes under nitrogen protection. The product was then dried in an oven with a vacuum of 23 mmHg and 38° C. for 26.1 hours to yield an loss on drying of 0.75%. Weight of t-butylaminoacetyl chloride HCl obtained=395 g, GC strength=89.5%, IR identification=positive. Yield from t-butylaminoacetic acid HCl=79%. Yield corrected for strength of product and starting material=71%.

### Example 22

### Tigecycline

9-Aminominocycline HCl (43.0 kg) was dissolved in 258 kg (6.0 parts water) for injection at 0 to 6° C. N-t-Butylglycine acid chloride HCl (47.3 kg, 1.1 parts, 3.01 equivalents) was added to the batch solution slowly while maintaining the batch temperature at 0 to 6° C. The reaction mixture was stirred for 1 h and determined to have 0.2% starting material (additional N-t-Butylglycine acid chloride HCl not required). The GAR-936 reaction mixture was then brought to pH 7.2+/−0.2 using 32 kg (0.7 parts) of ammonium hydroxide, 28%, and 2 kg reagent hydrochloric acid (to readjust overshoot). The initial pH equaled 0.42 and the final pH equaled 7.34. Methylene chloride (342 kg, 8 parts) and 148 kg (3.4 parts) methanol were added to the reaction mixture at 0 to 7° C. Since the pH was 7.09, no adjustment was required. The batch was warmed to 19 to 25° C. Methanol (83 kg, 1.9 parts) was added and the lower organic phase was split off. The product remaining in the aqueous phase was then extracted into the organic phase using 1×342 kg (8 parts) and 3×172 kg (4 parts) methylene chloride while maintaining pH at 7.2+/−0.2 with ammonium hydroxide, 28%. Methanol (49 kg, 1.14 parts) was added to the resulting methylene chloride/methanol solution, which was washed with 2×43 kg (1 part) water for injection before being dried with 43 kg (1 part) sodium sulfate. Three vacuum distillations were then performed to remove methanol with chases of 568 kg (13.2 parts) methylene chloride added prior to the second and third distillations. The residual level of methanol in the mother liquor was 0.21%. The batch was filtered, washed with 2×60 kg (1.4 parts) of pre-cooled (0 to 6° C.) methylene chloride. The resulting crude material was not dried, but isolated as a wet cake (72.5 kg, 38.2 kg dry weight as calculated from loss on drying), affording a 77% yield from 9-aminominocycline HCl. Wet cake analytical results: minocycline=1.26%, single largest impurity=0.37%, C-4 epimer=0.50%.

### Example 23

### Tigecycline

9-Aminominocycline HCl (61.0 kg) was dissolved in 258 kg (6.0 parts water) for injection at 0 to 6° C. N-t-Butylglycine acid chloride HCl (67.1 kg, 1.1 parts, 3.01 equivalents) was added to the batch solution slowly while maintaining the batch temperature at 0 to 6° C. The reaction mixture was stirred for 3.5 h and determined to have 0.13% starting material (additional N-t-Butylglycine acid chloride HCl not required). The reaction mixture was then brought to pH 7.2+/−0.2 using 45 kg (0.7 parts) of ammonium hydroxide, 28%. The initial pH equaled 0.82 and the final pH equaled 7.07. Methylene chloride (485 kg, 8 parts) and 210 kg (3.4 parts) methanol were added to the reaction mixture at 0 to 6° C. Since the pH was still in range (7.04), no adjustment was required. The batch was warmed to 19 to 25° C. Methanol (118 kg, 1.9 parts) was added and the lower organic phase was split off. The product remaining in the aqueous phase was then extracted into the organic phase using 1×485 kg (8 parts) and 3×244 kg (4 parts) methylene chloride while maintaining the pH at 7.2+/−0.2 with ammonium hydroxide, 28%. Methanol (70 kg, 1.14 parts) were added to the resulting methylene chloride/methanol solution, which was then washed with 2×61 kg (1 part) water for injection before being dried with 61 kg (1 part) sodium sulfate. Three vacuum distillations were then performed to remove methanol with chases of 805 kg (13.2 parts) methylene chloride added prior to the second and third distillations. The residual level of methanol in the mother liquor was 0.05%. The batch was filtered and washed with 2×85 kg (1.4 parts) of pre-cooled (0 to 6° C.) methylene chloride. The resulting crude material was not dried, but isolated as a wet cake (103 kg, 53.4 kg dry weight as calculated from loss on drying), affording a 76% yield from 9-aminominocycline HCl.

### Comparative Example 24

### Tigecycline Monohydrochloride

Example 24A: 9-chloroacetamidominocycline

Methylene chloride (1.3 L) was cooled to 0-2° C. in a 3-L round-bottom flask fitted with a mechanical, stirrrer, a thermometer and a 1-L addition-funnel. Recrystallized 9-aminominocycline hydrochloride (400 g) was added portion-wise with stirring. Triethylamine (428 mL) was added over 10 min. while keeping the temperature between 0-2° C. The reaction mixture was stirred for 10 min. and then cooled to −22° C. A solution of 280 g chloroacetic anhydride in 540 ml methylene chloride was then added at such a rate that the temperature did not rise above 5° C. An additional 132 ml of methylene chloride was used to rinse the addition funnel. The reaction mixture was assayed by HPLC 15 min after the start of anhydride addition. When the amount of starting material present was less than 2%, the reaction was quenched with 680 mL of 0.05M sodium bicarbonate solution. The mixture was stirred for 15 min, then transferred to a 5-L separatory funnel. The phases were allowed to separate. The methylene chloride phase was separated and washed with an additional 680 mL of 0.05M sodium bicarbonate solution. The washed methylene chloride solution was added dropwise into 17 L of a 10:1 mixture of n-heptane and isopropanol (15.4 L of n-heptane and 1.54 L of isopropanol). The slurry was stirred for 5 min. and then allowed to settle for 10 min. The supernatant was decanted off and the precipitate was filtered through a coarse-porosity fritted-funnel. The solid was washed with 2 L of 10:1 n-heptane:isopropanol. The solid was dried at 40° C. under vacuum to afford 550 g of the crude product.

Example 24B: Tigecycline

Crude 9-chloroacetamidominocycline (100 g) was added at room temperature (25-28° C.) slowly with efficient stirring to 500 mL of t-butylamine in a 1-L two-necked round-bottom flask fitted with a stirrer and thermometer. Sodium iodide (10 g) was added and the reaction mixture was stirred at room temperature for 7.5 h. The reaction was monitored by HPLC and when <2% starting material remained, 100 ml of methanol was added and the solvent was stripped off on a rotary evaporator at 40° C. To the residue was added 420 mL of methanol and 680 mL of water. The solution was cooled to 0-2° C. and adjusted to pH 7.2 with concentrated HCl (91 ml) to give a reaction mixture volume of 1300 mL. It was diluted to 6.5 L with water and the pH was adjusted to 4.0-4.2 with concentrated HCl (12 mL). Washed Amberchrom® (CG161cd) (860 g) was added to the solution and the mixture was stirred for 30 min., adjusting the pH to 4.0-4.2. The resin was filtered off and the spent aqueous solution was assayed by HPLC for product and stored at 4-8° C. The resin was slurried in 4.8 L of 20% methanol in water (4 L methanol+16 L water). The suspension was stirred for 15 min., adjusting pH 4.0-4.2. The resin was filtered off and the filtrate was assayed for product. The extraction of the resin was repeated 3 more times with 4.8 L of 20% methanol in water. All the resin extracts and the spent aqueous solution from above were pooled and the pH was adjusted to 7.0-7.2 with 30% ammonium hydroxide. The aqueous solution was extracted with 6×2.8 L of methylene chloride, adjusting the pH to 7.0-7.2 between extractions. The pooled methylene chloride extract was filtered through 250 g of anhydrous sodium sulfate, concentrated to 500 mL and cooled to 0-3° C. After the product crystallized, the slurry was stirred for 1 h at 0-3° C. The solids were filtered, washed with 2×50 mL of cold methylene chloride and dried at 40° C. under vacuum to afford 26 g of solid.

Example 24C: Tigecycline Monohydrochloride

Tigecycline (49 g, 0.084 mole) was dissolved portion-wise in 500 mL of water for injection with stirring. The solution was filtered through a medium porosity funnel and washed with 420 mL of water for injection. The solution was cooled to 0-2° C. and 5.6 mL of concentrated HCl was added dropwise while maintaining the temperature between 0-2° C. The initial pH was 8.0 and the final pH was 6.0. The solution was lyophilized by freezing the sample at −30° C. and lyophilizing at −15° C. The shelf temperature was raised to 21° C. for 2 h. The resulting solid (49.6 g) was ground and stored at 4-5° C. Elemental Analysis: C (52.92% theory, 51.75% found); H (6.73% theory, 6.75% found); N (10.65% theory, 10.32% found); Cl (5.4% theory, 5.5% found).

### Comparative Example 25

### Tigecycline Monohydrochloride

Example 25A: 9-chloroacetamidominocycline

Methylene chloride (325 mL) was cooled to −5 to 0° C. and 9-Aminominocycline hydrochloride (100 g) was added portion-wise over 10 min. Triethylamine (77.6 g) was added while maintaining the temperature at −10 to −5° C. A solution of 97% chloroacetic anhydride (70 g) in methylene chloride (133 mL) was prepared by stirring at 20-25° C. and added to the reaction mixture of 45 min while maintaining the mixture temperature at −10 to −2° C. The flask containing the chloroacetic anhydride solution was rinsed with 31 mL methylene chloride and the rinse added to the reaction mixture. After stirring for 30 min., the reaction was assayed by HPLC to determine if the reaction was complete. Aqueous sodium bicarbonate (185 mL, 0.05M) was added over 30 min while maintaining the reaction mixture temperature at 0 to 5° C. After stirring for 10 min., the layers were separated and sodium sulfate (15 g) was added to the organic layer. The mixture was stirred for 15 min at 0 to 5° C. and filtered. The resulting cake was rinsed with methylene chloride (2×38 mL) and the combined filtrates were transferred into 4.19 L of 10:1 heptane:isopropanol over 20 min, followed by a 15 mL methylene chloride rinse of the filtrate flask. The resulting suspension was stirred for 15 min at 20 to 25° C., then filtered. The cake was rinsed with 680 mL of 10:1 heptane:isopropanol and dried for 24 h at 37 to 40° C. (5-10 mm Hg). Purity by HPLC area %: 78.1.

Example 25B: Tigecycline

9-Chloroacetamidominocycline (100 g) was added with vigorous stirring to 483 mL of t-butylamine at 0-10° C. in a 2-L multi-necked round-bottom flask fitted with a stirrer, thermometer and condenser. Sodium iodide (16 g) was added and the reaction mixture was stirred at 33-38° C. for 4 h. The reaction mixture was assayed by HPLC for completion, then cooled to 5-10° C. Methanol (300 mL) was added over 10 min., then the reaction solution was concentrated by distillation (10-17° C., 68 mm Hg) to 350 mL. A second portion of methanol (600 mL) was added to the concentrate, and the mixture was concentrated by distillation to 350 mL. Methanol (46 mL) and cold water (565 mL) were added while maintaining the reaction temperature below 30° C. The solution was cooled to 0-5° C. and the pH adjusted to 4.0 with 100 mL of HCl 20° Be. The solution was transferred to a 5-L multi-neck flask with a 500 mL water rinse, then diluted with 1 L of water. After stirring for 1 h at 0-5° C., washed Amberchrom® (CG161) resin³ was added and the resulting suspension was stirred for 30 min. at 20-25° C. The suspension was filtered and the resulting wet cake was added to 340 mL of a 5:1 water:methanol solution. The filtrate was set aside. After stirring for 30 min. at 20-25° C., the suspension was filtered and the resulting wet cake was added to a second 340 mL portion of a 5:1 water:methanol solution. This second filtrate was set aside. This suspension was filtered and the resulting wet cake was added to a third 340 mL portion of a 5:1 water:methanol solution. After filtering, the third filtrate was combined with the first and second filtrates and cooled to 0-5° C. The pH was adjusted to 7.0 with 11 mL of 28% ammonium hydroxide. The solution was stirred at 0-5° C. for 16 h, adjusting the pH to 7.0 as necessary, and at 22-25° C. for 1 h, adjusting the pH to 7.0 as necessary. The aqueous solution was extracted with methylene chloride (5×980 mL), adjusting the pH to 7.0 for each extraction. The combined organic phases were transferred to a separatory funnel and the aqueous layer was separated. The organic layer was combined with 100 g sodium sulfate and stirred for 1 h at 20-25° C. The suspension was filtered through a celite pad and the cake was rinsed with 250 mL of methylene chloride. The filtrate was concentrated by distillation (−5 to 5° C., 150 mm Hg) to 150 mL, then cooled to 0-5° C. for 1 h. The resulting suspension was filtered and the cake was washed with 0-5° C. methylene chloride (2×30 mL). The wet cake was stirred in methylene chloride (335 mL) and methanol (37 mL) at 26-32° C. until a solution was obtained. The solution was filtered through celite, rinsing the celite with methylene chloride (2×15 mL), and concentrated by distillation (−5 to 5° C., 150 mm Hg) to 54 mL. The concentration procedure was repeated twice, first adding 335 mL methylene chloride and reducing the volume to 55-70 mL, then adding 254 mL methylene chloride and reducing the volume to 90-105 mL. The resulting suspension was stirred for 1 h at 0-5° C., then filtered and washed with −10° C. methylene chloride (2×25 mL). The solid was dried at 35-40° C. for 16 h, then at 45-50° C. for 27 h. Purity by HPLC area %: 97.7%, C-4 epimer 1.23%.
³ The washed Amberchrom® (CG161M) resin was prepared by adding 183 g of filtered, homnogonized Amberchrom® (CG161M) resin to 340 mL of a 5:1 water:methanol solution. After stirring for 1 h at 22-25° C., the suspension was filtered to give a wet cake that was dried by suction. The wet cake was stirred in 340 mL of a 5:1 water:methanol solution for 1 hr at 20° C., then filtered. The process was repeated once more to afford the washed resin.

Purification

### Example 1

### Tigecycline

A mixture of crude tigecycline (110.0 g) and methyl acetate (1.65 L) was stirred and heated to 30-35° C. and methanol (550 mL) was added over 15 min. After holding at 30-35° C., the warm solution was filtered over infusorial earth (36 g) and the cake was washed with methyl acetate (2×106 g). The filtrate was concentrated by distillation (20° C., 150 mm Hg) to 550 mL. Methyl acetate (1.1 L) was added and the resulting suspension was concentrated by distillation (20° C., 150 mm Hg) to 550 mL. This step was repeated, then the concentrate was cooled to 0-4° C. for 1 h. The resulting solid was collected by filtration and washed with 0-5° C. methyl acetate (2×150 mL). The solid was dried under vacuum (65-70° C., 10 mm Hg) for 100 h to afford 98.0 g (89.1% yield) of the desired product. Purity by HPLC area %: 98.8% and C-4 epimer 0.55%.

### Example 2

### Tigecycline

9-Aminominocyline.HCl (140.0 g) was added portion-wise to 840 mL of water at 0-4° C. t-Butylglycine acid chloride hydrochloride (154 g) was added over 15 min with good stirring while maintaining the temperature at 0-4° C. The solution was stirred for 1-3 h. The pH of the mixture was adjusted to 7.2±0.2 with 30% ammonium hydroxide while maintaining the temperature at 0-10° C. Methanol (930 mL) and 840 mL of methylene chloride were added to the solution, which was stirred for 1 h at 20-25° C. The phases were separated. The aqueous phase was extracted with 3×600 mL of methylene chloride, and the organic phases were combined, dried and concentrated to a volume of approximately 500 mL. The resulting suspension was cooled to 0-2° C. for 1 h. The solid was filtered and dried to give 120 g of product (75% yield). Purity by HPLC area %: 98%, C-4 epimer 0.1%. MS(FAB): m/z 586 (M+H); 585 (M⁺).

### Example 3

### Tigecycline

Tigecycline (15.00 g) prepared as described in Example 2 was added to 113 mL of acetone and 113 mL of methanol. The suspension was stirred at 20-25° C. for 1 h, then cooled to 0-2° C. After stirring for 1 h, the suspension was filtered and washed to give 12.55 g of product (83.7% yield). Purity by HPLC area %>99%, C-4 epimer 0.4%.

### Example 4

### Tigecycline

Tigecycline (105 g) prepared as described in Example 2 was added to 800 mL of acetone and 800 mL of methanol. The suspension was stirred and heated to 30-35° C. for 15 min, then cooled to 20-25° C. After holding at 20-25° C. for 1 h, the suspension was cooled to 0-4° C. and held for 1 h. The solid was filtered, washed and dried to give 83 g of product (79% yield). Purity by HPLC area %: >99%, C-4 epimer: 0.4%.

### Example 5

### Tigecycline

To a 1-L multi-necked flask, equipped with a mechanical stirrer and nitrogen protection, was added 94.3 g of wet crude tigecycline,⁴ methanol (305 g, 386 mL), and acetone (291 g, 368 mL). The mixture was stirred at 16-23° C. for 4 hours. The slurry was filtered on a 9-cm Buchner funnel with #1 Whatman paper. The wet cake was washed with methanol (87 g, 110 mL) at 20-25° C. The wet cake was dried with suction and nitrogen protection for 0.1 h. The wet cake (75.3 g) was transferred back to the 1-L multi-necked flask and a solution of methanol (233 g, 295 mL) and acetone (244 g, 309 mL) was added. The slurry was stirred at 15-20° C. for 5.5 hours. The slurry was filtered on a 9-cm Büchner funnel with #1 Whatman paper. The wet cake was washed with methanol (70 g, 88 mL) at 18-24° C. The wet cake was dried with suction and nitrogen protection for 0.1 h. The wet cake (59.0 g) was transferred back to the 1-L multi-necked flask and a solution of methanol (195 g, 247 mL) and acetone (187 g, 236 mL) was added. The slurry was stirred at 18-24° C. for 3 hours. The slurry was filtered on a 9-cm Büchner funnel with #1 Whatman paper. The wet cake was washed with methanol (55 g, 70 mL) at 20-25° C. The wet cake was dried with suction and nitrogen protection for 0.1 h. The wet cake (48.9 g) was sampled for high pressure liquid chromatography (HPLC) analysis (total impurities=0.62%, minocycline=0.17%, C-4 epimer=0.35%, largest other single impurity=0.05%).
⁴ Crude tigecycline was prepared from minocycline.HCl obtained from the supplier Interchem.

The wet cake (48.9 g) was transferred to a 2-L multi-neck flask with a vacuum distillation set up. To the wet cake was added a premixed solution of methanol (90 g, 114 mL) and dichloromethane (1,023 g, 772 mL). The slurry was stirred at 15-20° C. to obtain a red solution. The solution was distilled to 160 mL at 13-17° C. with a vacuum of 330 mmHg over 0.8 h to yield an orange slurry. To the 2-L flask was added dichloromethane (818 g, 617 mL) and the slurry was redistilled to 183 mL at 6-13° C. with a vacuum of 817 mmHg over 0.7 h. Dichloromethane (635 g, 479 mL) was added and the slurry redistilled to 183 mL at 6-7° C. with a vacuum of 817 mmHg over 0.6 hours. The resulting orange slurry was cooled to 0-5° C. and held at 0-5° C., with stirring, for 2 hours. The slurry was filtered on a 7-cm Büchner funnel with #1 Whatman paper. The wet cake was washed with two 69 g (52 mL) portions of dichloromethane at 0° C. The wet cake was dried with suction, under nitrogen protection, for 5 min. A sample of the wet cake (48.7 g) was submitted for HPLC analysis (total impurities=0.49%, minocycline=0.12%, C-4 epimer=0.32%, other impurities=0%.) The wet cake was then dried at 25° C. with a vacuum of <10 mmHg for 57.5 hours to a dichloromethane level of 2.2%, giving 32.3 g of tigecycline (34.2% yield).

This procedure was followed using crude Tigecycline that had been prepared from Minocycline.HCl obtained from suppliers Hovione and Nippon Kayaku. A comparison of the impurities present in the Tigecyline obtained from the above process using each source of Minocycline.HCl starting material is given in Tables 1 and 2. These tables indicate that the process provides a good yield of Tigecycline with a low level of impurities.

TABLE 1

| | Tigecycline | Total impurities | Recovered | C-4 epimer |
|---|---|---|---|---|
| | processing | in final | Minocycline•HCl | in final |
| Minoycline•HCl source | stage | Tigecycline (%) | (%) | Tigecycline (%) |
| Nippon Kayaku crude | crude | 0.71 | 0.33 | 0.26 |
| Nippon Kayaku purified | purified | 0.26 | 0.13 | 0.13 |
| Interchem crude | crude | 0.66 | 0.17 | 0.29 |
| Interchem purified | purified | 0.38 | 0.10 | 0.15 |
| Hovione crude | crude | 0.64 | 0.18 | 0.32 |
| Hovione purified | purified | 0.39 | 0.13 | 0.14 |

TABLE 2

| | Test | | |
|---|---|---|---|
| | Nippon Kayaku | Interchem | Hovione |
| Description | Orange powder | Orange powder | Orange powder |
| Yield (g) | 28.5 | 32.3 | 36.4 |
| Strength (%)¹ | 100 | 99.6 | 99.6 |
| Total impurities (%)² | 0.13 | 0.23 | 0.25 |
| LSI (%) [RRT]³ | brl⁴ | 0.13 [0.64] | 0.07 [0.67] |
| Minocycline (%) | 0.13 | 0.10 | 0.13 |
| Epimer (%) | 0.13 | 0.15 | 0.14 |
| Dichloromethane (%) | 1.3 | 2.2 | 2.1 |
| Methanol (%) | 0.001 | 0.003 | 0.002 |
| Acetone (%) | 0.001 | brl⁵ | brl |
| Heptane (%) | 0.001 | brl⁶ | brl |
| Isopropyl alcohol (%) | 0.002 | brl⁷ | brl |
| Toluene (ppm) | brl⁸ | brl | brl |
| N,N-Dimethyl- | brl⁹ | brl | brl |
| formamide (ppm) | | | |
| Water (KF, %) | 1.32 | 0.72 | 0.51 |
| Residue on ignition | 0.039 | 0.005 | 0.014 |
| (%) | | | |
| IR | Positive | Positive | Positive |
| Specific rotation¹ (°) | −219.4 | −213.4 | −218.7 |
| Crystallinity | Conforms | Conforms | Conforms |
| Yield (%) | 21 | 26 | 24 |
| Yield (corrected, %)¹⁰ | 24 | 30 | 27 |
| Yield from Mino (%) | 10 | 12 | 13 |
| Yield from Mino | 11 | 13 | 14 |
| (corrected, %)¹⁰ | | | |

¹On an anhydrous basis, solvent free.
²Excluding epimer.
³Largest single impurity (LSI) excluding C-4 epimer and Minocycline. Relative retention time (RRT) relative to GAR-936.
⁴brl: below reporting limit, 0.05% for HPLC.
⁵brl of 0.0005%.
⁶brl of 0.0003%.
⁷brl of 0.0030% (single sample).
⁸brl of 2 ppm.
⁹brl of 63 ppm.
¹⁰Corrected for strength of starting material and product.

### Example 6

### Tigecycline

Crude Tigecycline wet cake (72.5 kg, 38.2 kg dry weight⁵) was stirred and slurried in 191 kg (5 parts) acetone and 191 kg (5 parts) methanol. The slurry was then warmed to 30 to 36° C., immediately cooled to 19 to 25° C., and held at 19 to 25° C. for two hours. The slurry was then cooled to 0 to 6° C., and held at 0 to 6° C. for 1 hour. After filtering and washing with 2×34 kg (0.9 parts) acetone/methanol (1:1), the wet cake was then tested for minocycline (0.23%), 9-aminominocycline (0%), and for the largest single impurity other than the C-4 epimer (0.09%). The C-4 epimer content was 1.12%. Based on the analytical data, an additional reslurry was not performed. To the wet cake was added 440 kg (11.5 parts) of methylene chloride and 39.3 kg (1.0 parts) methanol and the mixture was heated to 30 to 36° C. to dissolve. The batch solution was filtered through 0.3-micron pyrogen reducing and 0.2-micron clarifying filters. Three vacuum distillations were then performed to remove methanol, with methylene chloride chases (440 kg and 339 kg, respectively) before the second and third distillations. The residual methanol level was 0.3%. The batch was cooled to 0 to 6° C. and stirred for 1 hour. The batch was filtered, washed with 2×42.1 kg (1.1 parts) of pre-cooled (−13 to −7° C.) methylene chloride and dried at no more than 60° C. to a loss on drying of <2.5%. The material was milled to give 22.3 kg of Tigecycline (58% yield). Purity by HPLC area %: 98.2%, C-4 epimer: 1.55%, Minocycline 0.1%, 9-aminominocycline 0%, single largest other impurity=0.08%.
⁵ Dry weight calculated form loss on drying data.

### Example 7

### Tigecycline

Crude Tigecycline wet cake (103.5 kg, 53.4 kg dry weight⁶) was stirred and slurried in 191 kg (5.1 parts) acetone and 191 kg (5.1 parts) methanol. The slurry was then warmed to 30 to 36° C., immediately cooled to 19 to 25° C., and held at 19 to 25° C. for two hours. The slurry was then cooled to 0 to 6° C., and held at 0 to 6° C. for 1 hour. After filtering and washing with 2×34 kg (0.9 parts) acetone/methanol (1:1), the wet cake was then tested for minocycline (0.12%), 9-aminominocycline (0%), and for largest single impurity other than C-4 epimer (0.13%). The C-4 epimer content was 0.37%. Based on analytical data, an additional reslurry was not performed. To the wet cake was added 440 kg (11.7 parts) of methylene chloride and 55.7 kg (1.0 parts) methanol and the mixture was heated to 30 to 36° C. to dissolve. The batch solution was filtered through 0.3-micron pyrogen reducing and 0.2-micron clarifying filters. Three vacuum distillations were then performed to remove methanol, with methylene chloride chases (624 kg and 481 kg, respectively) before the second and third distillations. The residual methanol level was 1.07%. The batch was cooled to 0 to 6° C. and stirred for 1 hour. The batch was filtered, washed with 3×59.7 kg (1.1 parts each) of pre-cooled (−13 to −7° C.) methylene chloride and dried at no more than 60° C. to a loss on drying of <2.5%. The material was milled to give 31.7 kg of Tigecycline as a first crop. A second crop consisting of residual product in the crystallizer provided an additional 2.5 kg. Both crops represent a 64% yield from crude Tigecycline.
⁶ Dry weight calculated form loss on drying data.

While the invention has been described by discussion of embodiments of the invention and non-limiting examples thereof, one of ordinary skill in the art may, upon reading the specification and claims, envision other embodiments and variations which are also within the intended scope of the invention and therefore the scope of the invention shall only be construed and defined by the scope of the appended claims.

What is claimed is:

1. A method of preparing at least one compound of formula 4, 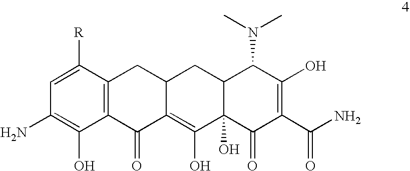
or a salt thereof,
wherein R=—NR₃R₄, where R₃ and R₄ are each independently chosen from hydrogen, and straight and branched chain (C₁-C₄)alkyl,
comprising:
combining at least one reducing agent with a reaction mixture slurry comprising an intermediate prepared from a reaction between at least one nitrating agent and at least one compound of formula 2, 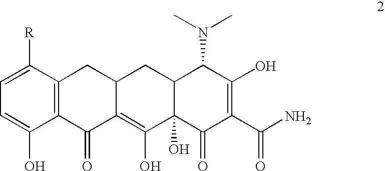
or a salt thereof.

2. The method according to claim 1, wherein the intermediate is a compound of formula 3 or a salt thereof.

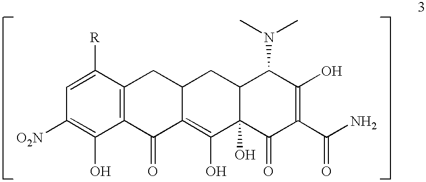

3. The method according to claim 1, wherein the at least one reducing agent is provided in the presence of at least one catalyst.

4. The method according to claim 3, wherein the at least one catalyst is chosen from rare earth metal oxides, Group VIII metal-containing catalysts, and salts of Group VIII metal-containing catalysts.

5. The method according to claim 4, wherein the at least one catalyst is chosen from Group VIII metal-containing catalysts.

6. The method according to claim 5, wherein the Group VIII metal-containing catalyst comprises palladium.

7. The method according to claim 6, wherein the Group VIII metal-containing catalyst is palladium on carbon.

8. The method according to claim 7, wherein the palladium on carbon catalyst is present in an amount ranging from 0.1 parts to 1 part, relative to the amount of the at least one compound of formula 2 present prior to the reaction with the at least one nitrating agent.

9. The method according to claim 1, wherein the at least one reducing agent is hydrogen.

10. The method according to claim 9, wherein the hydrogen is provided at a pressure ranging from 1 to 75 psi.

11. The method according to claim 10, wherein the hydrogen is provided at a pressure ranging from 1 to 50 psi.

12. The method according to claim 1, wherein prior to the combining, the reaction mixture is combined with a solvent comprising at least one (C₁-C₈) alcohol.

13. The method according to claim 12, wherein the at least one (C₁-C₈) alcohol is chosen from methanol and ethanol.

14. The method according to claim 1, wherein the combining is performed at a temperature ranging from 0° C. to 50° C.

15. The method according to claim 14, wherein the combining is performed at a temperature ranging from 20° C. to 40° C.

16. The method according to claim 15, wherein the combining is performed at a temperature ranging from 26° C. to 28° C.

17. The method according to claim 1, wherein the at least one nitrating agent is chosen from nitrate salts and nitric acid.

18. The method according to claim 1, wherein the at least one compound of formula 2 is a salt chosen from hydrochloride, hydrobromide, hydroiodide, phosphoric, nitric, sulfuric, acetic, benzoic, citric, cystein, fumaric, glycolic, maleic, succinic, tartaric, sulfate, and chlorobenzensulfonate salts.

19. The method according to claim 1, wherein after the combining, the reaction mixture contains the at least one compound of formula 2 in an amount less than 2% as determined by high performance liquid chromatography.

20. The method according to claim 1, wherein after the combining, the reaction mixture contains the at least one compound of formula 2 in an amount less than 1% as determined by high performance liquid chromatography.

21. The method according to claim 1, wherein after the combining, the reaction mixture is added to a solvent system comprising a (C₁-C₈) branched chain alcohol and a (C₁-C₈) hydrocarbon.

22. The method according to claim 21, wherein the (C₁-C₈) branched chain alcohol is isopropanol.

23. The method according to claim 21, wherein the (C₁-C₈) hydrocarbon is chosen from hexane, heptane, and octane.

24. The method according to claim 21, wherein after the combining, the reaction mixture is added to the solvent system at a temperature ranging from 0° C. to 50° C.

25. The method according to claim 21, wherein after the combining, the reaction mixture is added to the solvent system at a temperature ranging from 0° C. to 10° C.

26. The method according to claim 1, further comprising isolating the at least one compound of formula 4 as a solid composition.

27. The method according to claim 26, wherein the at least one compound of formula 4 is isolated as a salt.

28. The method according to claim 26, wherein the solid composition contains a C₄-epimer of formula 4 in an amount less than 10% as determined by high performance liquid chromatography.

29. The method according to claim 26, wherein the solid composition contains a C₄-epimer of formula 4 in an amount less than 1% as determined by high performance liquid chromatography.

30. The method according to claim 26, wherein the solid composition contains a C₄-epimer of formula 4 in an amount less than 0.5% as determined by high performance liquid chromatography.

31. The method according to claim 26, wherein the solid composition contains the at least one compound of formula 2 in an amount less than 2% as determined by high performance liquid chromatography.

32. The method according to claim 1, wherein the at least one compound of formula 2 is present in the reaction mixture in an amount of at least 1 gram.

33. A method of preparing at least one compound of formula 1, 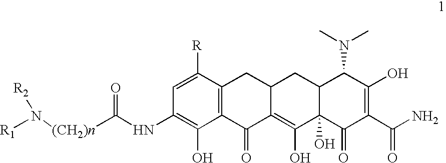
or a pharmaceutically acceptable salt thereof,
wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4,
comprising:
(a) combining at least one reducing agent with a reaction mixture slurry comprising an intermediate prepared from a reaction between at least one nitrating agent and at least one compound of formula 2, 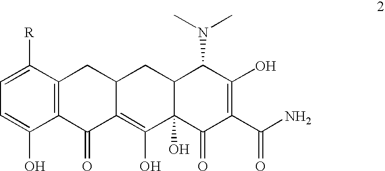
or a salt thereof, to form a second intermediate; and
(b) further reacting the second intermediate in the reaction mixture to prepare the at least one compound of formula 1.

34. The method according to claim 33, wherein R₁ is hydrogen, R₂ is t-butyl, R₃ is methyl, R₄ is methyl, and n is 1.

35. The method according to claim 34, wherein the at least one compound of formula 1 is tigecycline.

36. The method according to claim 33, wherein the second intermediate in the slurry is at least one compound of formula 4, 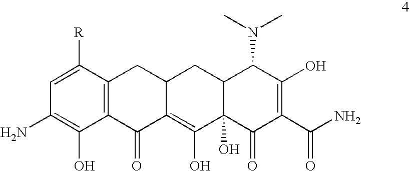
or a salt thereof.

37. The method according to claim 33, wherein the further reacting in (b) comprises acylating the second intermediate.

38. The method according to claim 37, wherein prior to the acylating, the second intermediate is isolated as a salt.

39. A method of preparing at least one compound of formula 4 or a salt thereof, 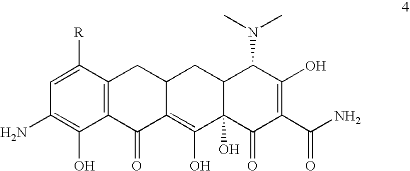
wherein R=—NR₃R₄, where R₃ and R₄ are each independently chosen from hydrogen, and straight and branched chain (C₁-C₄)alkyl,
comprising:
reducing an intermediate of formula 3 or a salt thereof, 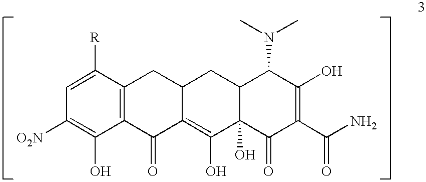
wherein the intermediate of formula 3 is present in a reaction mixture slurry.

40. The method according to claim 39, wherein the reducing comprises combining at least one reducing agent with the reaction mixture.

41. A method of preparing at least one compound of formula 1, 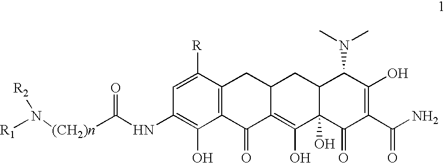
or a pharmaceutically acceptable salt thereof,
wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4,
comprising:
(a) reacting at least one nitrating agent with at least one compound of formula 2 or a salt thereof to prepare a reaction mixture, 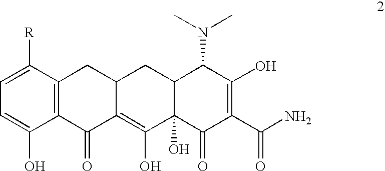
(b) without isolating any solids from the reaction mixture, combining at least one reducing agent with the reaction mixture to prepare an intermediate; and
(c) preparing the at least one compound of formula 1 from the intermediate.

42. The method according to claim 41, wherein R₁ is hydrogen, R₂ is t-butyl, R₃ is methyl, R₄ is methyl, and n is 1.

43. The method according to claim 42, wherein the at least one compound of formula 1 is tigecycline.

44. A method of preparing at least one compound of formula 1, 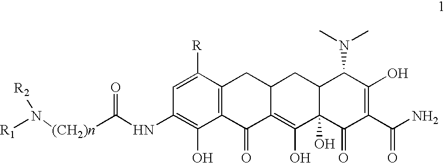
or a pharmaceutically acceptable salt thereof,
wherein R₁ and R₂ are each independently chosen from hydrogen, straight and branched chain (C₁-C₆)alkyl, and cycloalkyl, or R₁ and R₂, together with N, form a heterocycle; R is —NR₃R₄, where R₃ and R₄ are each independently chosen from hydrogen, and straight and branched chain (C₁-C₄)alkyl; and n ranges from 1-4,
comprising:
(a) combining at least one Group VIII metal-containing catalyst in the presence of hydrogen with a reaction mixture slurry prepared from a reaction between at least one nitrating agent and at least one compound of formula 2 or a salt thereof, 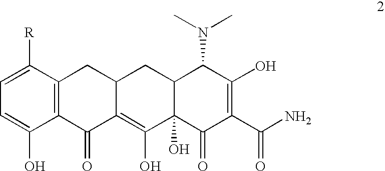
wherein the at least one Group VIII metal-containing catalyst is present in an amount ranging from 0.1 parts to 1 part relative to the amount of the at least one compound of formula 2 present prior to the reaction with the at least one nitrating agent.

45. The method according to claim 44, wherein R₁ is hydrogen, R₂ is t-butyl, R₃ is methyl, R₄ is methyl, and n is 1.

46. The method according to claim 45, wherein the at least one compound of formula 1 is tigecycline.

47. A compound or a salt thereof prepared by the method according to claim 1.

48. The compound according to claim 47, wherein R₃ is methyl and R₄ is methyl.

49. A compound or a salt thereof prepared by the method according to claim 33.

50. The compound according to claim 49, wherein R₁ is hydrogen, R₂ is t-butyl, R₃ is methyl, R₄ is methyl, and n is 1.

51. The compound according to claim 50, wherein the at least one compound of formula 1 is tigecycline.

52. The compound according to claim 51, wherein the at least one compound of formula 1 is tigecycline hydrochloride.

53. A compound or a salt thereof prepared by the method according to claim 39.

54. The compound according to claim 53, wherein R₃ is methyl and R₄ is methyl.

55. A compound or a salt thereof prepared by the method according to claim 41.

56. The compound according to claim 55, wherein R₁ is hydrogen, R₂ is t-butyl, R₃ is methyl, R₄ is methyl, and n is 1.

57. The compound according to claim 56, wherein the at least one compound of formula 1 is tigecycline.

58. The compound according to claim 57, wherein the at least one compound of formula 1 is tigecycline hydrochloride.

59. A compound or a salt thereof prepared by the method according to claim 44.

60. The compound according to claim 59, wherein R₁ is hydrogen, R₂ is t-butyl, R₃ is methyl, R₄ is methyl, and n is 1.

61. The compound according to claim 60, wherein the at least one compound of formula 1 is tigecycline.

62. The compound according to claim 61, wherein the at least one compound of formula 1 is tigecycline hydrochloride.

63. A composition comprising a compound or a salt thereof prepared by the method according to claim 1.

64. The composition according to claim 63, further comprising at least one pharmaceutically acceptable carrier.

65. The composition according to claim 63, wherein R₃ is methyl and R₄ is methyl.

66. A composition comprising a compound or a salt thereof prepared by the method according to claim 33.

67. The composition according to claim 66, further comprising at least one pharmaceutically acceptable carrier.

68. The composition according to claim 66, wherein R₁ is hydrogen, R₂ is t-butyl, R₃ is methyl, R₄ is methyl, and n is 1.

69. The composition according to claim 68, wherein the at least one compound of formula 1 is tigecycline.

70. The composition according to claim 69, wherein the at least one compound of formula 1 is tigecycline hydrochloride.

71. A composition comprising a compound or a salt thereof prepared by the method according to claim 39.

72. The composition according to claim 71, further comprising at least one pharmaceutically acceptable carrier.

73. The composition according to claim 71, wherein R₃ is methyl and R₄ is methyl.

74. A composition comprising a compound or a salt thereof prepared by the method according to claim 41.

75. The composition according to claim 74, further comprising at least one pharmaceutically acceptable carrier.

76. The composition according to claim 74, wherein R₁ is hydrogen, R₂ is t-butyl, R₃ is methyl, R₄ is methyl, and n is 1.

77. The composition according to claim 76, wherein the at least one compound of formula 1 is tigecycline.

78. The composition according to claim 77, wherein the at least one compound of formula 1 is tigecycline hydrochloride.

79. A composition comprising a compound or a salt thereof prepared by the method according to claim 44.

80. The composition according to claim 79, further comprising at least one pharmaceutically acceptable carrier.

81. The composition according to claim 79, wherein R₁ is hydrogen, R₂ is t-butyl, R₃ is methyl, R₄ is methyl, and n is 1.

82. The composition according to claim 81, wherein the at least one compound of formula 1 is tigecycline.

83. The composition according to claim 82, wherein the at least one compound of formula 1 is tigecycline hydrochloride.

84. A composition comprising:
at least one compound of formula 4, 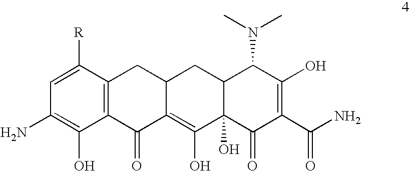
or a salt thereof,
wherein R is —NR₃R₄, where R₃ and R₄ are each independently chosen from hydrogen, and straight and branched chain (C₁-C₄)alkyl,
wherein a C₄-epimer of formula 4 is present in an amount less than 10%, as determined by high performance liquid chromatography.

85. The composition according to claim 84, wherein R₁ is hydrogen, R₂ is t-butyl, R₃ is methyl, R₄ is methyl, and n is 1.

86. The method according to claim 35, wherein the at least one compound of formula 1 is tigecycline hydrochloride.

87. The method according to claim 43, wherein the at least one compound of formula 1 is tigecycline hydrochloride.

88. The method according to claim 46, wherein the at least one compound of formula 1 is tigecycline hydrochloride.

89. The method according to claim 7, wherein the palladium on carbon is in wet form.

90. The method according to claim 27, wherein the at least one compound of formula 4 is isolated as a salt in the presence of sodium sulfite.
